(19) United States
(12) Patent Application Publication
(10) Pub. No.: US 2020/0000953 A1
(43) Pub. Date: Jan. 2, 2020

(54) METHOD FOR PRODUCING AROMA COMPOSITION FROM ANIMAL OR PLANT MATERIAL AND APPARATUS FOR COLLECTING AROMA FROM ANIMAL OR PLANT MATERIAL

(71) Applicant: T. HASEGAWA CO., LTD., Tokyo (JP)

(72) Inventors: Kazuhide ASHIKAGA, Fukaya (JP); Koutarou TANAKA, Fukaya (JP)

(21) Appl. No.: 16/452,723

(22) Filed: Jun. 26, 2019

(30) Foreign Application Priority Data
Jun. 29, 2018 (JP) 2018-124801

(51) Int. Cl. A61L 9/04

(52) U.S. Cl. CPC A61L 9/042; A61L 2209/22; A61L 9/046

(57) ABSTRACT

A method for producing an aroma composition from an animal or plant material, including: holding an adsorbent in a bag and putting the bag in a bag holder inside an aroma compound adsorbing device, fragmenting an animal or plant material to give crude fragmented pieces of the material containing minor fragments, removing the minor fragments from a gas containing aroma compounds emitted from the material in its fragmentation and minor fragments, introducing the gas from which the minor fragments have been removed into the adsorbent to adsorb the aroma compounds, taking out the bag from the bag holder, and collecting the aroma compounds from the adsorbent to prepare an aroma composition containing the aroma compounds, and the bag holder has a mesh lid at its both ends, and the bag has pores not passable by the adsorbent; the method is excellent in handleability of the adsorbent.

The present application claims the benefit of priority from Japanese Patent Application No. 2018-124801, filed on Jun. 29, 2018, the contents of which are herein incorporated by reference in their entirety.

### BACKGROUND OF THE INVENTION

### Technical Field of the Invention

The present invention relates to a method for producing an aroma composition from an animal or plant material and to an apparatus for collecting aroma from an animal or plant material.

### Description of the Related Art

An aroma composition is used as a food flavoring or a fragrance. An aroma composition for food and drink can be prepared from a natural flavorings, synthetic chemicals for flavorings and/or a flavoring composition of a combination of the former two, and with the recent tendency toward consumer needs for naturalness, a flavoring is also desired to be derived from natural materials or a flavoring having a feel of nature, and various production methods are now under investigation.

Various animal and plant materials are used for flavor production. Taking coffee as an example, various production methods are now employed for coffee flavorings. Among coffee flavorings, in particular, those capable of giving a freshly ground coffee aroma are desired for long. Accordingly, a method of using an aroma that is emitted in grinding roasted coffee beans is proposed. Here, JP 6184627 B1 discloses, as an already-existing technique, a method of introducing a gas (ground gas) that contains an aroma component emitted in grinding roasted coffee beans, directly into a solvent of water, a coffee oil or the like followed by storing it in an aluminum container with compression under pressure therein.

As opposed to this, JP 6184627 B1 proposes a method for producing an aroma composition from roasted coffee beans, which includes a step of grinding roasted coffee beans to give a crude ground product of roasted coffee beans containing a fine powder and thin flakes, and includes a step of removing the fine powder and thin flakes from a gas that contains aroma compounds emitted from the roasted coffee beans in grinding the roasted coffee beans and contains the fine powder and thin flakes, an adsorbing step of introducing the gas from which the fine powder and thin flakes have been removed into an adsorbent to thereby make the aroma compounds adsorbed by the adsorbent, and a collecting step of collecting the aroma compounds from the adsorbent to prepare an aroma composition containing the aroma compounds, and in which the adsorbent is held in an adsorbent holder in an aroma compound adsorbing device, and the adsorbent holder has a mesh lid at both ends thereof in the gas flowing direction therethrough.

### SUMMARY OF THE INVENTION

JP 6184627 B1 describes, in Examples therein, a method of directly holding an adsorbent inside a sidewall-type basket not having pores through the sidewall thereof, as an adsorbent holder. Using the device in Examples in JP 6184627 B1, the present inventors investigated a method of collecting aroma compounds by introducing a gas flow from which a fine powder and others have been removed into an adsorbent. As a result, the inventors have found that, in order to take as much as possible of the adsorbent having adsorbed aroma compounds out of the adsorbent holder for the purpose of collecting a large amount of the aroma compound, the gas-applied adsorbent scatters as charged with static electricity, and therefore, it is difficult to take out an almost whole amount of the adsorbent having adsorbed the aroma compound, and have found another problem that much time is taken for taking out the adsorbent.

An object of the present invention is to provide a method for producing an aroma composition from an animal or plant material which can collect an aroma emitted in fragmenting the animal or plant material and which is excellent in handleability of an adsorbent having adsorbed aroma compounds. The subject matter for this object is newly recognized by aiming to efficiently take out as much as possible of the adsorbent through which an aroma compound-containing gas is introduced.

The present inventors have made assiduous studies for the purpose of solving the above-mentioned problems. As a result, the inventors have found that, when a gas from which a fine powder and others have been removed is introduced into an adsorbent while the adsorbent is kept held in a bag such as a mesh bag, and when the adsorbent is, together with the bag that is holding the adsorbent therein, taken in and out of an aroma compound adsorbing device (e.g., a column), then the adsorbent can be prevented from being electrostatically charged and from scattering owing to the charging with static electricity, and therefore a nearly whole amount of the adsorbent having adsorbed aroma compounds can be taken out, and further the time necessary for taking out the adsorbent can be shortened and the process efficiency can be thereby improved.

This is a method not heretofore known in the technical field of collecting gaseous aroma compounds from an animal or plant material.

Here, in a field of exhaust treatment and purification technology where a gaseous matter that contains undesired substances (such as gaseous or solid contaminants) is introduced into a container holding an adsorbent therein in an upflow fluidized-bed manner so as to make the undesired substances trapped by the adsorbent, there is known a method of holding an adsorbent in a gas-passable bag (see JP H06-306377 A). JP H06-306377 A describes a method for removing malodorous components from a fuel gas, wherein a fuel gas that contains mercaptans as malodorous components is brought into contact with hydrogen and/or a polyvalent metal ion-exchange zeolite except alkaline earth metals in an oxygen-free atmosphere. However, in JP H06-306377 A, zeolite is merely held in a sample bag and a city gas is introduced into the sample bag in confirming the malodor-adsorbing performance of zeolite, that is, such a sample bag is merely used as a test holder for holding an adsorbent therein in place of an adsorbent container such as a column. In addition, in JP H06-306377 A, nothing is referred to relating to a technique of facilitating taking in and out of an adsorbent to thereby take out a nearly whole amount of an adsorbent having adsorbed aroma compounds.

On the other hand, in the case where not a gaseous but a liquid matter is introduced into an adsorbent or the like for collecting valuable substances, it is known to hold an adsorbent in a liquid-passable bag (see JP 2013-133987 A). JP 2013-133987 A describes a drier for a freezing cycle equipped with an absorbent that can absorb water contained in a coolant, in which the absorbent is a polyacrylic acid or polyacrylate-based polymer having a specific structure, and when a relationship between a relative humidity in an atmosphere around the adsorbent and a rate of water adsorption by the adsorbent is plotted, the water adsorption rate increment increases with the increase in the relative humidity. However, in JP 2013-133987 A, a bag is used merely because there is no other means for holding the adsorbent in view of the structure of the trapping device therein.

The present invention as a specific means for solving the above-mentioned problems and preferred embodiments thereof are as described below.

[1] A method for producing an aroma composition from an animal or plant material, comprising:

a holding step of holding an adsorbent in a bag and putting the bag in a bag holder inside an aroma compound adsorbing device,

a step of fragmenting an animal or plant material to give crude fragmented pieces of the animal or plant material that contain minor fragments,

a step of removing the minor fragments from a gas that contains aroma compounds emitted from the animal or plant material in fragmenting the animal or plant material and contains the minor fragments,

an adsorption step of introducing the gas from which the minor fragments have been removed into the adsorbent to thereby make the aroma compounds adsorbed by the adsorbent,

a takeout step of taking out the bag from the bag holder, and

a collecting step of collecting the aroma compounds from the adsorbent to prepare an aroma composition containing the aroma compounds, wherein:

the bag holder has a mesh lid at both ends thereof in the gas flowing direction therethrough, and

the bag has pores in a size through which the adsorbent could not pass.

[2] The method for producing an aroma composition from an animal or plant material according to [1], wherein the adsorbent is one or more selected from a styrene-divinylbenzene copolymer, an ethylvinylbenzene-divinylbenzene copolymer, a 2,6-diphenyl-9-phenyl oxide polymer, a condensation polymer of a methacrylic acid and a diol, and a modified silica gel.
[3] The method for producing an aroma composition from an animal or plant material according to [1] or [2], wherein the gas flowing direction is substantially an opposite direction to the direction of gravitational force.
[4] The method for producing an aroma composition from an animal or plant material according to [3], wherein the bag is provided with a weight and the weight is arranged around the peripheral part at the end of the gas inlet side of the bag.
[5] The method for producing an aroma composition from an animal or plant material according to any one of [1] to [4], wherein the end part of the gas outlet side of the bag is drawn and gathered.
[6] The method for producing an aroma composition from an animal or plant material according to any one of [1] to [5], wherein:

the bag holder has a gas direction adjuster on the gas inlet side of the bag, and the gas is introduced into the inside of the bag via the gas direction adjuster.

[7] The method for producing an aroma composition from an animal or plant material according to any one of [1] to [6], wherein the adsorbent is in the state of a fluidized bed in the adsorption step.
[8] The method for producing an aroma composition from an animal or plant material according to any one of [1] to [7], wherein the aroma compounds are desorbed from the adsorbent using an organic solvent in the collection step.
[9] The method for producing an aroma composition from an animal or plant material according to any one of [1] to [8], wherein the adsorbent is kept held in the bag in the collecting step.

An apparatus for collecting aroma from an animal or plant material, which is provided with:

a device for fragmenting an animal or plant material,

a first flow channel which is communicated with the fragmenting device and through which a gas can flow, together with aroma compounds contained in the gas and minor fragments contained in the gas, which aroma compounds have been emitted in fragmenting the animal or plant material,

a minor fragments removing device communicated with the first flow channel,

a second flow channel which is communicated with the minor fragments removing device and through which the gas, from which the minor fragments have been removed, can flow,

an aroma compound adsorbing device communicated with the second flow channel, and

a gas flow generating device that generates a gas flow continuing from the fragmenting device to the aroma compound adsorbing device,

a bag capable of holding an absorbent, and in which:

the aroma compound adsorbing device has a bag holder capable of detachably holding the bag,

the bag holder has a mesh lid at both ends thereof in the gas flowing direction therethrough, and

the bag has pores in a size through which the adsorbent could not pass.

[11] The apparatus for collecting aroma from an animal or plant material according to [10], wherein the gas flowing direction is substantially an opposite direction to the direction of gravitational force.
[12] The apparatus for collecting aroma from an animal or plant material according to [11], wherein the bag is provided with a weight and the weight is arranged around the peripheral part at the end of the gas inlet side of the bag.
[13] The apparatus for collecting aroma from an animal or plant material according to any one of [10] to [12], wherein the end part of the gas outlet side of the bag is drawn and gathered.
[14] The apparatus for collecting aroma from an animal or plant material according to any one of [10] to [13], wherein:

the bag holder has a gas direction adjuster on the gas inlet side of the bag, and

the gas is introduced into the inside of the bag via the gas direction adjuster.

[15] The apparatus for collecting aroma from an animal or plant material according to any one of [10] to [14], wherein the adsorbent is held in a bag.

According to the present invention, there can be provided a method for producing an aroma composition from an animal or plant material, which can collect an aroma emitted in fragmenting the animal or plant material and which is excellent in handleability of an adsorbent having adsorbed aroma compounds.

Also, according to the present invention, there can be provided an apparatus for collecting an aroma from an animal or plant material, which can collect an aroma emitted in fragmenting the animal or plant material and which is excellent in handleability of an adsorbent having adsorbed aroma compounds.

### BRIEF DESCRIPTION OF THE DRAWINGS

FIG. 1 is a schematic view showing an example of an aroma collecting apparatus of the present invention.

FIG. 2 is a schematic view showing another example of an aroma collecting apparatus of the present invention.

FIG. 3 is a cross-sectional schematic view of an example of an aroma compound adsorbing device usable for the present invention.

FIG. 4 is a cross-sectional schematic view of another example of an aroma compound adsorbing device usable for the present invention.

FIG. 5 is a cross-sectional schematic view of another example of an aroma compound adsorbing device usable for the present invention.

FIG. 6(A) is a perspective schematic view of an exemplary state of an aroma compound adsorbing device where a bag is provided without a weight. FIG. 6(B) is a perspective schematic view of an exemplary state of an aroma compound adsorbing device where a bag is provided with a weight around the peripheral part at the gas inlet side end of the bag. FIG. 6(C) is a perspective schematic view of an exemplary state of an aroma compound adsorbing device having a gas direction adjuster. FIG. 6(D) is a perspective schematic view of an exemplary state of an aroma compound adsorbing device not having a gas direction adjuster.

FIGS. 7(A) and (B) each are a schematic plan view of an example of a bag for use in the present invention. FIG. 7(C) is a perspective schematic view of a bag for use in the present invention, exemplifying a mode of holding a weight in the bag. FIG. 7(D) is a bottom view of one example of a mode where a weight is held in a bag for use in the present invention.

FIG. 8 is a graph showing a relationship between the lapse time from the start of gas introduction (adsorbing time) and the pressure or the gas flow at different sites in an aroma collecting apparatus, in the adsorbing step in Example 1.

FIG. 9 is a graph showing a relationship between the lapse time from the start of gas introduction (adsorbing time) and the pressure or the gas flow at different sites in an aroma collecting apparatus, in the adsorbing step in Example 2.

FIG. 10 is a graph showing a relationship between the lapse time from the start of gas introduction (adsorbing time) and the temperature at a column inlet port and a column outlet port, in the adsorbing step in Example 1.

FIG. 11 is a graph showing a relationship between the lapse time from the start of gas introduction (adsorbing time) and the temperature at a column inlet port and a column outlet port, in the adsorbing step in Example 2.

### DESCRIPTION OF EMBODIMENTS

The present invention is described in detail hereinunder. The description of the constitutive elements of the invention given hereinunder is for some typical embodiments or examples of the invention, to which, however, the invention should not be limited. In this description, the numerical range expressed by the wording “a number to another number” means the range that falls between the former number indicating the lower limit of the range and the latter number indicating the upper limit thereof.

[Method for Producing Aroma Composition from Animal or Plant Material]

The method for producing an aroma composition from an animal or plant material of the present invention (hereinafter this may be referred to as the production method of the present invention) includes:

a holding step of holding an adsorbent in a bag and putting the bag in a bag holder inside an aroma compound adsorbing device,

a step of fragmenting an animal or plant material to give crude fragmented pieces of the animal or plant material that contain minor fragments,

a step of removing the minor fragments from a gas that contains aroma compounds emitted from the animal or plant material in fragmenting the animal or plant material and contains the minor fragments,

an adsorption step of introducing the gas from which the minor fragments have been removed into the adsorbent to thereby make the aroma compounds adsorbed by the adsorbent,

a takeout step of taking out the bag from the bag holder, and

a collecting step of collecting the aroma compounds from the adsorbent to prepare an aroma composition containing the aroma compounds, wherein:

the bag holder has a mesh lid at both ends thereof in the gas flowing direction therethrough, and

the bag has pores in a size through which the adsorbent could not pass.

Having the constitution as above, the present invention can provide a method for producing an aroma composition from an animal or plant material, which can collect an aroma emitted in fragmenting the animal or plant material and which is excellent in handleability of an adsorbent having adsorbed aroma compounds. In particular, in the case where a gas containing aroma compounds is introduced into an adsorbent, the adsorbent can be taken in and out of an aroma compound adsorbing device together with a bag that is holding the adsorbent therein, and consequently, the adsorbent can be prevented from being electrostatically charged and from scattering owing to the charging with static electricity, and therefore as much as possible of the adsorbent can be taken out, and further the time necessary for taking out the adsorbent can be shortened and the method is excellent in processability and efficiency.

In the case where a gas containing aroma compounds is introduced into a fluidized-bed state of an adsorbent in an upflow direction (substantially in a vertical upward direction), the adsorbent may be more readily electrostatically charged and may scatter owing to the charging with static electricity. On the other hand, the production method of the present invention can prevent the adsorbent from being electrostatically charged and from scattering owing to the charging with static electricity, and therefore according to the method, as much as possible of the adsorbent can be taken out, and further the time necessary for taking out the adsorbent can be shortened.

According to the preferred embodiment of the present invention, an aroma composition that gives an aroma emitted and perceivable in fragmenting an animal or plant material can be preferably produced from an animal or plant material using an ordinary fragmenting device and without requiring any additional great capital investment and serious load on equipments. Further, according to the production method of the present invention, preferably, an aroma composition can be produced from an animal or plant material, which aroma composition can give not only an aroma emitted and perceivable in fragmenting the animal or plant material at the top but also a mild and voluminous flavor and a good aftertaste in the middle and later to various foods and drinks, or can enhance such an aroma or flavor of various foods and drinks.

When an animal or plant material is fragmented into a desired size, in addition to fragmented pieces in a desired size, there are formed any one or more of fragmented pieces and thin flakes derived from the animal or plant material which do not meet the desired size and fragmented pieces and thin flakes derived from any other foreign substances (in this description, these are collectively referred to as “minor fragments”), and the minor fragments are light and scatter. In industrial fragmentation of an animal or plant material, at least a part of the minor fragments scatter and mix in an exhaust gas flow that is generated from the fragmenting device. Heretofore, the exhaust gas has been discharged out of the device as it is, after the minor fragments have been appropriately removed therefrom.

Here, in the present invention, an adsorbent held in a bag that is arranged in a bag holder inside an aroma compound adsorbing device is used in place of a solvent (liquid) for collecting aroma compounds. If an exhaust gas containing minor fragments therein is, as it is, directly introduced into an adsorbent, it is considered that the minor fragments may clog a mesh lid and may also clog fine pores of an adsorbent and even fine voids between adsorbent particles to make the exhaust gas difficultly flow therethrough so that the exhaust system of the fragmenting device will be overloaded (pressured). As opposed to this, the production method of the present invention employs a method where, after minor fragments have been removed from the exhaust gas, the resultant exhaust gas is introduced into an adsorbent to make the adsorbent adsorb aroma compounds, and according to the production method of the present invention, therefore, the aroma compounds contained in the exhaust gas can be adsorbed by the adsorbent with no risk of clogging of the mesh lid and the adsorbent and no risk of giving a load on the device. In addition, owing to this clogging prevention, the aroma compounds can be efficiently adsorbed by the adsorbent.

Referring to exhaust system performance of an ordinary fragmenting device, when an whole bag with adsorbent inside is held in an bag holder arranged in the flow channel of an exhaust gas flow in the device, a load over an allowable range may be given to the device owing to the resistance of the adsorbent to the exhaust gas flow (in this description, this may be simply referred to as a load). Consequently, a means of suppressing the resistance owing to the adsorbent may be employed. For example, the length in the flowing direction of an exhaust gas flow (also referred to as the gas flow direction) in the part occupied by the adsorbent held in the bag holder (hereinafter in this description, this may be referred to as an adsorbent part, or a held adsorbent part) is reduced; or a flow channel that is branched from the flow channel of an exhaust gas flow and holds an adsorbent therein is arranged so as to collect aroma compounds from a part of the exhaust gas. Apart from these exemplifications, the resistance of adsorbent may also be suppressed by enhancing the mobility of the adsorbent held in the device (for example, using a so-called “fluidized-bed column”). Further, a blower or a suction pump may be additionally used for ventilation of the adsorbent over the resistance thereof.

Preferred embodiments of the production method of the present invention are described below.

### <Holding Step>

The production method of the present invention includes a holding step of holding an adsorbent in a bag and putting the bag in a bag holder inside an aroma compound adsorbing device.

### (Adsorbent)

In the holding step, an adsorbent is held in a bag. The method for holding an adsorbent in a bag is not specifically limited.

In the case where an adsorbent has an extremely small water content, the adsorbent may crack owing to an impact given thereto in its transportation depending on the material of the adsorbent, resulted in decreasing the adsorption efficiency of the adsorbent. In such a case, for preventing the adsorbent from cracking, it is desirable that the adsorbent is made to absorb water (preferably pure water) and then, before completely dried, the adsorbent is held in a bag.

The adsorbent amount is not specifically limited so far as it can be held in a bag. The volume (bulk volume) of the adsorbent to be used may be the same as or less than the volume of the bag holder and the bag. In other words, the adsorbent may be filled up in a bag holder and a bag (roughly or densely), or the bag holder or the bag that holds an adsorbent may have some vacant space (namely, a part not occupied by the adsorbent). From the viewpoint of adsorption efficiency and reduction in the load on an aroma collecting apparatus and for the purpose of enhancing the mobility of the adsorbent to form a state of a fluidized-bed column during the process of gas introduction into the adsorbent, it is desirable that the volume of the bag and the bag holder is larger than the volume of the adsorbent.

The adsorbent is not specifically limited. The adsorbent usable herein includes a synthetic adsorbent, and any other adsorbent such as an inorganic adsorbent such as active carbon, silica gel, zeolite, magnesia or titania. A synthetic adsorbent is preferably used from the viewpoint of easiness in desorption and reuse. A reuse method will be described hereinunder.

Preferably, in the present invention, the adsorbent is one or more selected from a styrene-divinylbenzene copolymer, an ethylvinylbenzene-divinylbenzene copolymer, a 2,6-diphenyl-9-phenyl oxide polymer, a condensation polymer of a methacrylic acid and a diol, and a modified silica gel. The modified silica gel is a chemically-bonded silica gel prepared by chemically bonding a reactive substance such as an alcohol, an amine, a silane or the like to the surface of a silica gel by utilizing the reactivity of the silanol group with the reactive substance. Above all, a styrene-divinylbenzene copolymer is preferred.

The adsorbent is preferably a resin, more preferably a porous polymer resin. The surface area of the adsorbent is, for example, preferably about 300 m²/g or more, more preferably about 500 m²/g or more. Also preferably, the pore size distribution of the adsorbent is about 10 A to about 500 A.

Not specifically limited, the shape of the adsorbent is granular. Also not specifically limited, the average particle diameter of the granular adsorbent may be, for example, within a range of 0.1 to 20 mm, or 0.1 to 1 mm.

Examples of the porous polymer resin satisfying the above-mentioned requirements include an HP resin (manufactured by Mitsubishi Chemical Corporation), an SP resin of a styrene-divinylbenzene copolymer (manufactured by Mitsubishi Chemical Corporation), and XAD-4 (manufactured by DowDuPont Inc.), and these are readily available on the market. Also, commercial products of a methacrylate resin, for example, XAD-7 and XAD-8 (manufactured by DowDuPont Inc.) are also available.

Preferred examples of the SP resin include Sepabeads SP-70 and SP-207.

### (Bag)

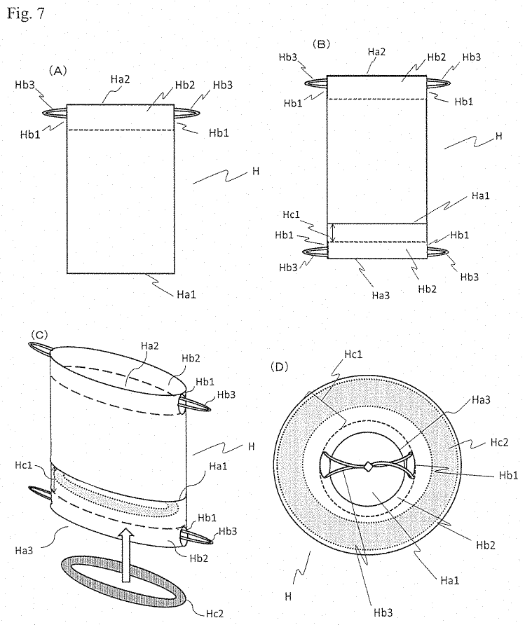

In the holding step, a bag that is holding an adsorbent therein is put in a bag holder inside an aroma compound adsorbing device. The method of putting the bag in a bag holder is not specifically limited. The bag may be merely placed inside a bag holder, but from the viewpoint of preventing any unnecessary movement or deformation of the bag owing to gas flow, preferably, at least a part of the bag is detachably fixed and put in a bag holder. More preferably, one end of the bag is detachably fixed to a bag holder. For example, employable is a method of arranging one or more hook-like, string-like or loop-like members at one end of the bag as hooking means, and hooking them on catching members attached to a bag holder or an aroma compound adsorbing device. As one example, there may be mentioned a method of hooking strings arranged to pass through a string passage having stringing holes in one end part of the bag (hereinafter this may be referred to as a gas outlet side end, including, for example, the end on the gas outlet side—) (see FIG. 7 to be mentioned below), on any desired part of a bag holder or an aroma compound adsorbing device and fixing it thereon. On the other hand, the other end part of the bag (hereinafter this may be referred to as a gas flow side end, including, for example, the end on the gas inlet side-) may be or may not be fixed to the bag holder or the aroma compound adsorbing device, but is preferably not fixed thereto from the viewpoint that the bag can be readily detached. Also in any other method, a bag may be put in a bag holder.

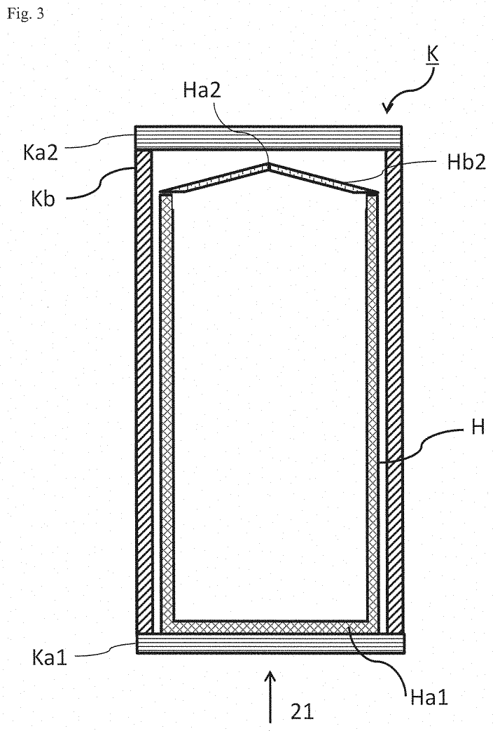

The gas inlet side end of the bag kept held in a bag holder may be kept in contact with or not contact with a mesh lid Ka1 to be mentioned below (see FIGS. 3 to 5 to be mentioned below). In the case of no contact therebetween, the bag is kept hung, probably resulted in a decrease in the area of the cross section perpendicular to the gas flowing direction through the bag (hereinunder unless otherwise specifically indicated, the cross section perpendicular to the gas flowing direction through the bag is merely referred to as a cross section) owing to the weight of the adsorbent so that the flowability of the adsorbent would lower, and depending on the size of the cross-sectional area, the flow resistance could increase or the gas flow could hardly spread entirely over the adsorbent. Accordingly, the bag is preferably so dimensioned that the bottom of the bag can reach the bottom of the bag holder and the bag can inflate through gas introduction thereinto so as to be in contact with the inner wall of the bag holder, thereby securing high flowability of the adsorbent, and this embodiment is desirable from the viewpoint of adsorption efficiency.

In the present invention, the bag has gas-passable pores in a size through which a gas can pass but the adsorbent cannot. The number of pores is preferably larger as facilitating gas passage therethrough. Preferably, the bag is meshed, and so far as gas passage through the bag can be kept in a desired state, the bag may have an unmeshed part (gas-impassable part), but preferably the bag is entirely meshed.

The bag of the type can prevent the adsorbent held in the bag from leaking out of an aroma compound adsorbing device and enables gas introduction through the adsorbent. The opening of the meshed bag may be any ones as long as not permitting leakage of the adsorbent therethrough. One example of the mesh opening is, though not limited thereto, a range of 10 μm to 20 mm, preferably 50 μm to 5 mm, more preferably 100 μm to 2 mm.

The volume of the bag is not also specifically limited, and may be 50% or more, 70% or more, 80% or more, 90% or more, or 95% or more of the volume of the bag holder, or may also be around the same as the volume of the bag holder, but as so mentioned hereinabove, from the viewpoint of aroma compound adsorption efficiency, the bag is preferably so dimensioned that the bottom of the bag can reach the bottom of the bag holder and the bag can inflate through gas introduction thereinto so as to be in contact with the inner wall of the bag holder.

The configuration of the bag is not specifically limited. The bag has any arbitrary configuration so far as the bag can hold an adsorbent therein, the adsorbent held therein does not leak out while the gas flowing therethrough, and the bag has one or more closable mouths for an entrance of the adsorbent put therethrough in and out the bag.

FIGS. 7(A) and (B) each are a schematic plan view of an example of a bag for use in the present invention. The bag H has a gas inlet side end Ha1 and a gas outlet side end Ha2. Preferably, the bag is so configured that the gas inlet side end Ha1 is closed, and the gas outlet side end Ha2 is openable and closable. From the mouth at the gas outlet side end Ha2, an adsorbent may be put into the bag, and the adsorbent may be kept inside the bag by the closed gas inlet side end Ha1.

In the present invention, when the bag is held in a bag holder, which is to be mentioned below, in a holding step the gas outlet side end Ha2 of the bag may be drawn and gathered toward the center of the cross section of the bag perpendicular to the gas flowing direction therethrough, and the other part of the bag may be tubular, for example, cylindrical (see FIGS. 3 to 5 to be mentioned below). For example, the bag H in FIG. 7(A) has a string passage Hb2 at the gas outlet side end Ha2. The string passage Hb2 is formed by sewing indicated with the dotted line and has stringing holes Hb1. The string passage Hb2 acts as a channel for a string Hb3 passing therethrough. In FIG. 7(A), an adsorbent is held in the bag H, and then the strings Hb3 coming out from the two stringing holes Hb1 at the gas outlet side end Ha2 are drawn to gather the gas outlet side end Ha2 to thereby close the bag to form a shape like a drawstring bag or pouch. Using a bag having such a configuration, the shape of the bag H in the gas outlet side end Ha2 can be so formed that the side of the bag H is drawn and gathered toward the center of the cross section of the bag, while the other part of the bag is kept tubular (for example, cylindrical) (see FIGS. 3 to 5). Accordingly, the adsorbent can be prevented from scattering out of the bag through the gas outlet side end Ha2, and in addition, since the bag can be opened or closed by loosening or tightening the strings, the workability in the holding step and also the bag handleability in the subsequent steps can be bettered.

The adsorbent entrance mouth of the bag (e.g., the gas outlet side end Ha2 in FIG. 7) may be opened and closed by, in place of the above-mentioned strings, any other means such as buttons, fasteners, or hook and loop fasteners (so-called Magic Tape (registered trademark)), and is preferably so configured that it is easy to open and close and the adsorbent held therein does not leak out.

The material of the bag includes metals and resins, and resins are preferred. Examples of the resins include polyesters, polypropylenes, polyethylenes, Teflon (registered trademark), and nylons. The bag may be formed of the same material as a whole, or a part thereof may be formed of a different material.

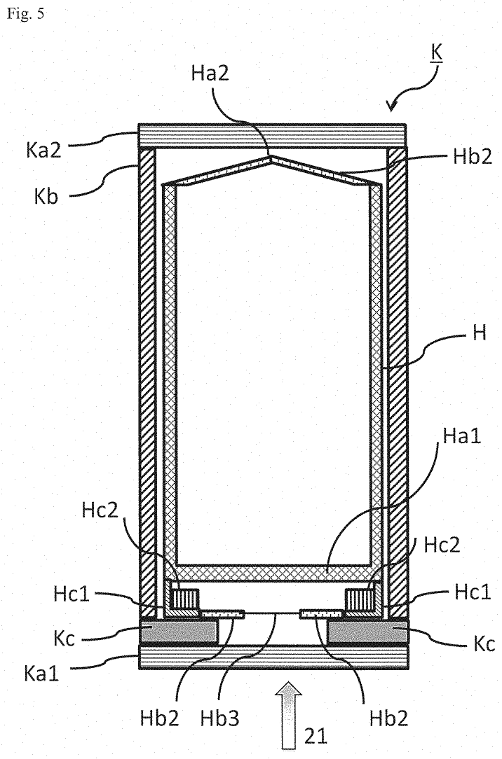

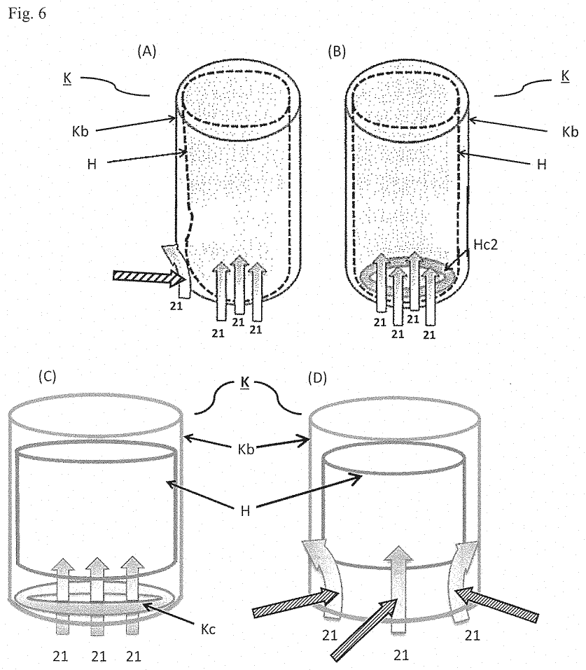

The bag preferably has a configuration capable of being provided with a weight (also referred to as a weight holding part) in the gas inlet side end thereof. From the viewpoint of not interfering with gas flowing into the bag, more preferably, such a weight structure is arranged around the peripheral part at the gas inlet side end of the bag (see FIG. 5, FIG. 6(B) and FIG. 7(B) to (D)).

In the present invention, preferably, the bag kept held in the bag holder in the holding step is provided with a weight around the peripheral part at the gas inlet side end. In particular, it is desirable that the gas inlet side end is arranged to be on the lower side in the vertical direction and a weight is arranged around the peripheral part at the gas inlet side end. When the bag has such a weight around the peripheral part at the gas inlet side end, the efficiency in introducing a gas that contains aroma compounds emitted from an animal or plant material in fragmenting the animal or plant material (in other words, the gas from which minor fragments have been removed) into the bag can be enhanced and the aroma compound adsorption efficiency can be thereby enhanced. In particular, in the adsorbing step where much force of the gas to deform the bag is applied owing to the influence of the flow rate of the gas that contains aroma compounds and of the gas flow direction thereinto, the bag provided with a weight at the gas inlet side end, especially around the peripheral part thereof, can prevent the cross-sectional area of the bag from being reduced by the gas flow. Further, when the weight and the periphery of the bag (size and the shape of the periphery) are made to be equal to those of the bag holder and are so configured that the weight is provided around the peripheral part of the bag, the bag can be readily in contact with the inner side wall of the aroma compound adsorbing device, thereby preventing the bag from being unstably lifted up in the bag holder.

The effect of the weight holding part and the weight will be more specifically described with reference to FIG. 6(A) and (B).

FIG. 6(A) is a perspective schematic view of an exemplary state of an aroma compound adsorbing device where a bag is provided without a weight (where an aroma compound-containing gas is introduced into the device with no weight). FIG. 6(B) is a perspective schematic view of an exemplary state of an aroma compound adsorbing device where a bag is provided with a weight around the peripheral part at the gas inlet side end (at the end in the perpendicular lower direction) (where an aroma compound-containing gas is introduced into the device provided with such a weight). Mesh lids Ka1 and Ka2 are not shown in these Figures. The embodiments of FIGS. 6(A) and (B) are examples where the peripheries of the bag holder, the bag and the weight are nearly the same.

In the case where the bag H is provided without a weight, as shown in FIG. 6(A), a part of the bag H may possibly be deformed (for example, lifted up) owing to the pressure of the gas flow and a part of the gas (the gas flow as marked by the hatched arrow in FIG. 6(A)) would be led outside the bag. On the other hand, in FIG. 6(B), by virtue of the bag H provided with a weight Hc2 around the peripheral part on the gas inlet side, much of or preferably the whole of the gas that contains aroma compounds 21 can be introduced into the inside of the bag H, and in addition, the gas can be made to flow uniformly through an adsorbent (not shown) held in the bag H to thereby enhance the efficiency of collecting the aroma compounds 21.

The bag H exemplified in FIG. 7(B) is further provided with a weight holding part Hc1 around the peripheral part at the gas inlet side end Ha1. The bag H exemplified in FIG. 7(B) is, in addition to the weigh holding part Hc1, further provided with a string passage Hb2 positioned on the gas inlet side of the weight holding part Hc1, and in the case where no weight is held therein, the edge of the string passage Hb2 (the end of the gas inlet side) is opened and functions as a weight taking-in and taking-out mouth Ha3. In the bag H exemplified in FIG. 7(B), the string passage Hb2 positioned on the gas inlet side of the weight holding part Hc1 has two stringing holes Hb1. On the other hand, the gas inlet side end Ha1 of the bag H is closed like in the bag of FIG. 7(A), and the adsorbent put into the bag via the gas outlet side end Ha2 is kept inside the bag H at the gas inlet side end Ha1.

FIG. 7(C) is a perspective schematic view of the bag H provided with the weight holding part Hec1, exemplifying a mode of holding the weight Hc2 in the bag H. The bag H of FIG. 7(C) is cylindrical, and has an openable gas outlet side end Ha2, a closed gas inlet side end Ha1, and a weight holding part Hc1 and a string passage Hb2 extending from the periphery at the edge of the gas inlet side end Ha1. The weight taking-in and taking-out mouth Ha3 is openable and closable by the strings Hb3 like that on the gas outlet side end Ha2. In putting the weight Hc2 into the bag, for example, the weight Hc2 is put close to the gas inlet side end Ha1 via the weight taking-in and taking-out mouth Ha3, and then the strings Hb3 are drawn to narrow the weight taking-in and taking-out mouth Ha3 to thereby set the weight Hc2 in the bag. FIG. 5 and FIG. 7(D) exemplify this condition.

FIG. 5 is a schematic view of a cross section along the gas flowing direction of another example of an aroma compound adsorbing device for use in the present invention (an example provided with a bag holder that holds a bag with a weight held therein). FIG. 7(D) is a bottom view of the bag H, exemplifying an example of a state where a weight Hc2 is held in the weight holding part Hc1. When a weight Hc2 having nearly the same periphery as that of the bag is put via the weight taking-in and taking-out mouth Ha3 in the direction toward the inside of the bag and when the two strings Hb3 passing through the string passage Hb2 at the gas inlet side and coming out from the stringing holes Hb1 are drawn and tightened, then the gas inlet side of the string channel Hb2 is drawn and gathered to form a shape like a drawstring bag or pouch and the weight holding part Hc1 is to have a lid-like structure, and with that, the weight Hc2 is held inside the weight holding part Hc1. Specifically, by drawing and tightening the strings Hb3, the weight holding part Hc1 is folded toward the center along the periphery of the weight Hc2 so that the weight Hc2 is mounted on a part of the weight holding part Hc1 that is nearly parallel to the cross section of the bag (see especially FIG. 5). In FIG. 7(D), the outline of the weight Hc2 is shown by a long-dotted line, and this means that the weight Hc2 held under the condition exists on the reverse side of the outside of the weight holding part Hc1 that is visible in this viewpoint. The lid-like structure of the case can be opened or can be substantially closed so far as the weight Hc2 can be held stably, and FIG. 5 and FIG. 7(D) are examples where the lid-like structure is opened, in which the outer bottom face of the gas inlet side end Ha1 of the bag is seen through the opening of the structure.

Also in the case of the bag having the configuration as above, it is desirable that the whole of the bag H including the weight holding part Hc1 and the string passage Hb2 has gas-passable pores in a size through which a gas can pass but the adsorbent held in the bag cannot. More preferably, the whole of the bag H is meshed. The whole bag may be formed of the same material, or a part of the bag may be formed of a different material.

Having the structure, the bag maintains a high gas flowing efficiency through the bag for a gas that contains aroma compounds (in other word, a gas from which minor fragments have been removed), and a weight can be readily attached to and detached from the bag.

Needless-to-say, the structure of the weight holding part Hc1 is not limited to the above-mentioned cases. For example, in place of the holding means for the weight Hc2 in FIG. 7(B) to (D) in which the strings Hb3 passing through the string passage Hb2 are drawn to narrow the end part of the bag for deformation, buttons, hooks, fasteners, hook and loop fasteners (so-called Magic Tape (registered trademark)) or the like may be arranged in the weight holding part Hc1, and the end side of the bag may be drawn and deformed by these to thereby make the bag have a structure capable of holding the weight Hc2 therein. Alternatively, a hooking means such as a hook or a loop may be arranged in the gas inlet side end Ha1, and the weight Hc2 may be hung thereon or may be detachably fixed thereto. Depending on the characteristics (shape, material, mass) of the weight to be used, the weight holding means may be designed in any desired manner.

The weight is not specifically limited. The material of the weight includes metals and resins, and metals are preferred. One example of the metals for the weight is stainless steel.

The mass of the weight is not specifically limited, and is preferably such that the bag is not deformed by the force of a gas flow.

The shape of the weight is not also specifically limited. In the case of the weight holding part Hc1 in FIG. 7(B) to (D), the weight Hc2 may have a frame-like structure having a hole in the center thereof and having any desired shape (for example, a square frame, an oval having a hole in the center, a disc having a concentric hole in the center (“doughnut” shape)). As exemplified in FIG. 6(B) and FIGS. 7(C) and (D), preferably, the frame-like weight Hc2 has a hole in the center thereof, the outer periphery of the weight Hc2 is nearly the same as the periphery of the bag H and the bag holder Kb, and the frame width is as small as possible. In such a configuration, the cross section of the gas inlet side end Ha1 is prevented from being narrowed by a gas flow therethrough, and in addition, the weight can reduce its gas flow disturbance and blocking, and consequently, the configuration of the type is preferred from the viewpoint of enhancing the gas flowing efficiency through the bag for a gas that contain aroma compounds and enhancing the aroma compound adsorption efficiency.

Also from the same viewpoint, plural weights may be arranged symmetrically around the peripheral part at the gas inlet side end. In addition, weights may be arranged in any other sides than the peripheral part at the gas inlet side end.

For preventing the adsorbent from cracking, preferably, the bag is held in the bag holder inside the aroma compound adsorbing device before the adsorbent having adsorbed water (preferably pure water) and put into the bag is completely dried.

The amount of the adsorbent to be held in the bag is not specifically limited, and is preferably such an amount as to be a volume (bulk) smaller than the volume of the bag capable of holding the adsorbent therein (this may be simply referred to as a bag volume) in order that the adsorbent inside the bag could be in a fluidized-bed state during gas introduction thereinto. The volume of the adsorbent relative to the bag volume is not specifically limited, but is preferably within a range of 5% to 80% relative to the bag volume, more preferably 10% to 50%. For example, the range may be such that the fluidized-bed column can be kept as such for a predetermined period of time (for example, 1 hour or more) within the range, and the maximum value in the range may be employed.

### (Bag Holder)

FIG. 3 is a schematic view of the cross section along the gas flowing direction) of an example of an aroma compound adsorbing device for use in the present invention. The aroma compound adsorbing device K in FIG. 3 is provided with a bag holder Kb, a mesh lid Ka1 on the gas inlet side and a mesh lid Ka2 on the gas outlet side, and this shows a state where the bag H is held in the holding step. In this, by drawing and tightening the strings (not shown) passing through the string passage on the gas outlet side end Ha2, the gas outlet side end Ha2 is closed. The bag H is has a meshed structure having pores in a size through which a gas can pass but an adsorbent cannot, and consequently, even when the gas outlet side end Ha2 is closed, a gas that contains aroma compounds 21 can pass through the gas outlet side end Ha2. On the other hand, the pores (opening of the mesh) of the bag H are in such a size through which an adsorbent cannot pass and in addition, the gas outlet side end Ha2 is closed. Consequently, the adsorbent held in the bag H does not scatter out of the bag H in the adsorbing step. The strings may be hung on the hooking means (not shown) in the aroma compound adsorbing device K.

In a state where the bag with an adsorbent held therein is held in the bag holder, the bag holder may have a vacant space not occupied by the adsorbent, or may not have such a space. Preferably, the bag holder has such a space in the gas flowing direction from the viewpoint that, during gas introduction in the adsorbing step, the adsorbent part can readily be in a state of a fluidized bed. Specifically, it is desirable that the bag holder in the aroma compound adsorbing device contains a part occupied by the adsorbent held in the bag (adsorbent part) and a vacant space part not occupied by the adsorbent.

The space between the bag holder and the bag, as mentioned hereinabove, is desirably as small as possible, in other words, the bag is kept in contact with the inner wall surface of the bag holder as much as possible, from the viewpoint of preventing turbulence flow generation to be caused by gas introduction between the bag holder and the outer side face of the bag to thereby enhance the gas introduction efficiency into the adsorbent, which gas contains aroma compounds (in other words, a gas from which minor fragments have been removed) and to enhance the aroma compound adsorption efficiency (see FIG. 6(A)).

After the bag holding step, the length of the adsorbent part in the gas flowing direction in a static state before and after the adsorbing step (namely, in a state with no gas introduction) is not specifically limited, but from the viewpoint of reducing the resistance of the adsorbent, the length is preferably 1000 mm or less, more preferably 700 mm or less, even more preferably 500 mm or less, further more preferably 400 mm or less, still more preferably 300 mm or less, and especially more preferably 200 mm or less. For example, the length of the adsorbent part may be within a range of 10 mm to 800 mm, or 20 mm to 600 mm, or 30 mm to 500 mm, or 40 mm to 400 mm, or 50 mm to 300 mm.

After the bag holding step, the major axis or the diameter (hereinafter for convenience sake, collectively referred to as a diameter) of the face perpendicular to the gas flowing direction of the adsorbent part (hereinafter this may be simply referred to as a cross section of the adsorbent part) in a static state before and after the adsorbing step (namely, in a state with no gas introduction) is not specifically limited, but is preferably controlled depending on the amount of the adsorbent and the length of the adsorbent part. From the viewpoint of easiness in gas passage therethrough, the diameter of the cross section of the adsorbent part is preferably 10 mm or more, more preferably 30 mm or more, even more preferably 50 mm or more, still more preferably 100 mm or more, further more preferably 200 mm or more, and especially more preferably 300 mm or more.

In increasing the adsorbent amount, it is desirable to increase the cross-sectional diameter of the adsorbent part to reduce the length in the gas flowing direction of the adsorbent part from the viewpoint of reducing the resistance of the adsorbent to the gas flow.

Though not specifically limited, the bag holder may be, for example, a basket. As the basket, there are known a normal-type basket having pores through the side surface thereof and a sidewall-type basket not having pores through the side surface thereof. Using a sidewall-type basket not having pores through the side surface thereof is preferred from the viewpoint that the gas from which minor fragments have been removed would not leak out through the pores of the side surface of the basket and therefore the length of the gas flowing through the adsorbent can be increased.

### —Mesh Lid—

In the present invention, the bag holder has a mesh lid at both ends in the gas flowing direction therethrough. The mesh lid prevents the bag held in the bag holder from leaking out of the aroma compound adsorbing device and enables the gas to flow through the adsorbent.

The mesh lid is a sheet or a thin plate having a desired thickness, and, not specifically limited thereto, the size thereof can be selected to fall within a range capable of preventing the adsorbent from leaking out of the aroma compound adsorbing device. From the viewpoint of securing easy gas flowing therethrough, the mesh lid preferably has an area not less than the cross-sectional area of the bag holder. The mesh lid may partly or wholly be meshed. From the viewpoint of securing easy gas flowing therethrough, preferably, a part of the mesh lid corresponding to the cross section of the aroma compound adsorbing device or the bag holder has a mesh structure.

The opening of the mesh lid may be selected as long as the adsorbent used may not pass therethrough. Not limited thereto, an example of the opening is within a range of 10 μm to 20 mm.

### —Gas Direction Adjuster—

In the present invention, preferably, the bag holder has a gas direction adjuster on the gas inlet side thereof, and through the gas direction adjuster, more gas can be introduced into the bag than without the gas direction adjuster. This is because, when a larger amount of a gas that contains aroma compounds is introduced into the inside of the bag, then the amount of the adsorbent in contact with the aroma compound-containing gas can be increased.

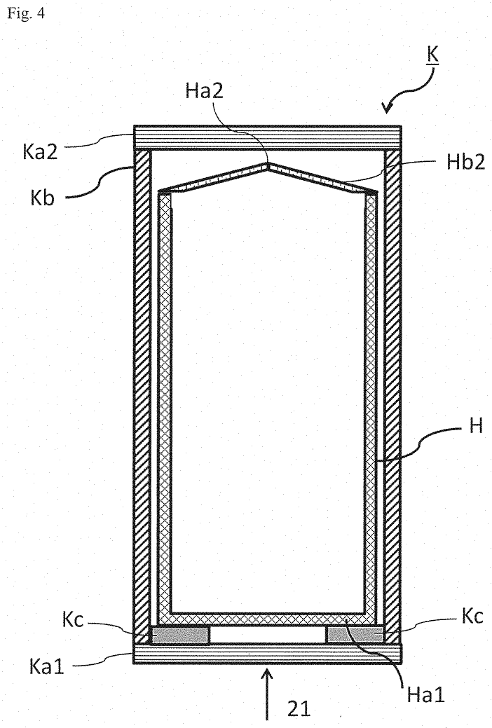

FIG. 4 is a schematic view of the cross section along the gas flowing direction) of another example of an aroma compound adsorbing device for use in the present invention. In FIG. 4, a gas direction adjuster Kc is arranged on the gas inlet side end of the bag holder Kb.

The gas direction adjuster is not specifically limited so far as it can make a larger amount of a gas flow into the inside of the bag. For example, a baffle plate is preferred. A baffle plate is a flow restraining plate to be arranged in a gas flow line (also referred to as a turning blade), and can change a gas flow direction.

The material of the baffle plate includes metals and resins, and metals are preferred. One example of the metals is stainless steel.

The shape of the baffle plate is not specifically limited. For example, it may be a tabular or cylindrical member, or may be a frame-like member having a hole in the center and having any desired shape (for example, a square frame, an oval having a hole in the center, a disc having a hole in the center (so-called doughnut shape)). The member of the type can change the gas flow direction along the plate-like or cylindrical shape thereof. In addition, the baffle plate can control the gas direction to flow more within the hole.

In the case of a frame-structured baffle plate having a hole in the center, preferably, the diameter of the hole is smaller than the diameter of the bag in a state of gas introduction thereinto in the adsorbing step. The diameter of the hole in the center of the baffle plate is smaller by 0.1 to 20.0% than the diameter of the cross section of the bag in a state of gas introduction thereinto in the adsorbing step, and is more preferably smaller by 1 to 10.0%. Having such a configuration, a gas that contains aroma compounds 21 can more readily flow through the inside of the bag H and the aroma compound collection efficiency can be thereby enhanced.

FIG. 5 is a more preferred embodiment where the bag H is provided with a weight Hc2 in the aroma compound adsorbing device of FIG. 4 (in this, an adsorbent is not shown). For the weight holding part Hc1 and the condition of holding the weight Hc2 therein, refer to the above-mentioned description and FIGS. 7(C) and (D).

As shown in FIG. 5, especially preferably, the aroma compound adsorbing device K is provided with a gas direction adjuster Kc on the gas inlet side, and the bag H is provided with a weight Hc2 around the peripheral part of the gas inlet side end Ha1.

As described hereinabove, the bag H in FIG. 5 holds the weight Hc2 in the peripheral part at the gas inlet side end Ha1. On the cross-sectional view, by drawing and tightening the strings Hb3, the string passage Hb2 and the weight holding part Hc1 are drawn toward the center of the bag H as shown in FIG. 7(C), and the weight holding part Hc1 is deformed along the weight Hc2, and as a result, the weight Hc2 is mounted on the weight holding part Hc1 as illustrated in the view. In FIG. 5, the weight holding part Hc1 are kept in contact with the gas direction adjuster Kc, but there may be some gap between the weight holding part Hc1 and the gas direction adjuster Kc. Preferably, the weight holding part Hc1 and the gas direction adjuster Kc are kept in contact with each other since the gas that contains aroma compounds 21 can more readily flow into the inside of the bag H in such a condition.

In the case where the weight Hc2 is held in the bag, the inner diameter of the gas direction adjuster Kc is preferably smaller than the inner diameter of the weight Hc2. In the case where the weight Hc2 is used, preferably, a hole of the gas direction adjuster Kc is arranged in such a manner that the gas flow could flow into the part where the gas flow is not blocked by the weight Hc2, as illustrated in FIG. 5 (in FIG. 5, the hole is arranged inside than the weight Hc2, that is, in the center side of the bag H).

The positional relationship between the gas direction adjuster, the mesh lid and the bag holder are not specifically limited. For example, in the embodiment of FIG. 4, the gas direction adjuster is held inside the bag holder, but as in the embodiment of FIG. 5, the gas direction adjuster may be sandwiched between the bag holder and the mesh lid.

FIG. 6(C) is a perspective schematic view of an example of an aroma compound adsorbing device having a gas direction adjuster Kc (in which mesh lids Ka1 and Ka2 are not shown). In the example shown in FIG. 6(C), the gas direction adjuster Kc is a doughnut-shaped baffle plate, and three arrows each indicate a flow of a gas containing aroma compounds 21 and having passed through the hole of the gas direction adjuster Kc. In FIG. 6(C) provided with the gas direction adjuster Kc, the gas that contains aroma compounds 21 can readily flow into the inside of the bag H and the aroma compound collection efficiency can be thereby enhanced.

On the other hand, FIG. 6(D) is a perspective schematic view of an example of an aroma compound adsorbing device not having a gas direction adjuster (in which mesh lids Ka1 and Ka2 are not shown). In the case not having a gas direction adjuster, a part of the gas that contains aroma compounds 21 flow into the outside of the bag H, and the flow of the gas that contains aroma compounds 21 in FIG. 6(D) (see the arrows marked by hatched arrows) becomes a turbulence flow while the cross-sectional area of the bag H is reduced, and as a result, the aroma compound collection efficiency in this case would probably be reduced as compared with that in FIG. 6(C).

### <Step of Preparing Crude Fragmented Pieces of Animal or Plant Material>

The production method of the present invention includes a step of fragmenting an animal or plant material to give crude fragmented pieces of the animal or plant material that contain minor fragments.

Preferably, the step of fragmenting an animal or plant material to give crude fragmented pieces of the animal or plant material is carried out after holding step and prior to the other step.

In this description, “fragmenting” means that an animal or plant material are processed into small fragments having a desired size according to any arbitrary method of grinding, fracturing, mincing, powdering or flaking. In the fragmenting step, minor fragments that are derived from the animal or plant material or foreign substances and are smaller than the desired size are generally formed.

The method of fragmenting an animal or plant material is not specifically limited, and any known method is employable. For example, any arbitrary apparatus capable of fragmenting an animal or plant material, for example, a grinding device such as a roller mill, a jet mill, a hammer mill, a rotary mill or a shaking mill, as well as a flaking machine or a powdering machine may be used in accordance with the intended object.

Not specifically limited, the fragmenting speed for an animal or plant material may be set arbitrarily according to the type of animal or plant material, for example, 1 to 500 kg/h.

Also not specifically limited, the fragmenting size of an animal or plant material may be set arbitrarily according to the type of animal or plant material. For example, the size may be 0.08 to 3 mm or so.

The animal or plant material to be processed in the fragmenting step may have an original size at the time when they are obtained, but may be pre-fragmented to have a size larger than the intended size.

### (Animal or Plant Material)

Not specifically limited, the animal or plant material for use in the production method of the present invention may be any ones capable of being fragmented and capable of being used for production of any arbitrary products such as foods and drinks, cosmetics, health and hygiene products and medicines.

The animal or plant material may be any one capable of being fragmented to be drunk or eaten as such, or may be any one capable of being used for production of any arbitrary products such as foods and drinks, cosmetics, health and hygiene products and medicines. Preferred examples thereof include roasted animal and plant materials. Specifically, examples of the animal or plant material include, though not limited thereto, roasted or unroasted coffee beans, roasted or unroasted cacao nuts, nuts (peanuts, almonds, cashew nuts, walnuts, etc.), tea (roasted green tea, powdered green tea, etc.), dried products (dried animal materials such as dried small sardines, and dried plant materials such as dried mushrooms), dried fishes (various dried bonitos), other dried fishes than dried bonitos (dried Souda bonitos, dried mackerels, dried tunas, dried round herrings, dried sardines, dried sauries, etc.), buckwheat, spices (pepper, thyme, capsicum, cinnamon, turmeric, etc.), sesames, soybeans, lavers, and herbs.

For example, the animal and plant materials include may be roasted coffee beans. Not adhering to any theory, it is presumed that the kind of coffee beans and the roasting level of coffee may mainly influence the mass ratio of the aroma compound(s) having a large molecular weight in an aroma composition. The aroma that is emitted in grinding roasted coffee beans is an aroma at the top (to be caused by a volatile aroma compound(s) having a small molecular weight), and therefore it is presumed that the kind of coffee beans and its roasting level would have little influence on the aroma. Consequently, the present invention is applicable to multi-purpose utilization, not depending on the kind and the roasting level of coffee beans.

The coffee beans for use in the production method of the present invention may be, for example, any of Arabica coffee, Robusta coffee, or Liberica coffee, and any coffee beans are employable herein irrespective of kind and production area thereof. Raw coffee beans may be roasted in any ordinary method using a coffee roaster or the like. For example, raw coffee beans are put into a rotary drum, and with rotating the rotary drum for stirring, the beans may be heated from the below with a gas burner or the like to be roasted. The roasting level is generally expressed by L value, 16 to 19 for Italian roast; 19 to 21 for French roast; 21 to 23 for Full city roast; 23 to 25 for City roast; 25 to 27 for High roast; and 27 to 29 for Medium roast. Softer roasting than these is not so much used for ordinary coffee. The L value is an index that indicates the level of coffee roasting, and is a value of the lightness of a ground powder of roasted coffee beans measured with a colorimeter. Black is represented by an L value 0, and white is by 100. Accordingly, harder roasted coffee beans have a lower value, and softer roasted coffee beans have a higher value.

The kind of coffee beans, the roasting method for coffee beans and the treatment method for roasted coffee beans are not specifically limited. For example, the methods described in [0015] to [0027] in JP 2013-252112 A, and [0021] to [0024]in JP 2015-149950 A may be employed. The contents of these patent publications are incorporated herein by reference.

### (Crude Fragmented Pieces of Animal or Plant Material)

Preferably, the crude fragmented pieces of animal or plant material contain the above-mentioned minor fragments, and fragmented pieces of an animal or plant material fragmented into a desired size.

Preferably, the minor fragments are removed from the gas that contains aroma compounds emitted from an animal or plant material in fragmenting the animal or plant material. Specifically, it is preferable that the minor fragments pass through a first flow channel to be mentioned in detail hereinunder, along with the gas, and are removed from the gas in a minor fragments removing device.

### (Aroma Compounds Emitted in Fragmenting Animal or Plant Material)

The aroma compound that is emitted from an animal or plant material in fragmenting the animal or plant material includes one or plural compounds.

### <Step of Preremoving Minor Fragments>

In the production method of the present invention, preferably, a step of removing minor fragments from the crude fragmented pieces of an animal or plant material is carried out prior to the step of removing minor fragments from the gas mentioned above. The minor fragments may be partly or substantially wholly removed partly.

The step of removing minor fragments from the crude fragmented pieces of an animal or plant material may be carried out using any known minor fragments removing device, for example, a classification device such as a shaking sieve or a wind classification device. A classification device using a shaking sieve is preferred. For example, using a sieve having a desired opening, minor fragments smaller than the opening may be removed.

For example, in the case where the roasted coffee beans are used, preferably, a step of removing a fine powder and thin flakes from the crude ground powder of roasted coffee beans is carried out prior to the step of removing a fine powder and thin flakes from the gas mentioned above. The fine powder and thin flakes may be removed partly, but substantially the whole thereof may be removed. Also, a fine powder and thin flakes derived from any others than chaff may be mainly removed, or a fine powder and thin flakes derived from chaff may be removed at least partly in the preremoving step, or almost all thereof may not be removed. This preremoving step can further reduce the load on the aroma collecting apparatus (in particular, to the exhaust system).

### <Step of Removing Minor Fragments>

The production method of the present invention includes a step of removing minor fragments from a gas that contains aroma compounds emitted from an animal or plant material in fragmenting the animal or plant material and contains minor fragments. Not removed, the minor fragments may partly remain, but preferably, substantially the whole of the minor fragments are removed.

The step of removing minor fragments is not specifically limited, and may be carried out in any known method.

For example, in the case where the roasted coffee beans are used, the production method includes a step of removing a fine powder and thin flakes from a gas that contains an aroma compound that is emitted from roasted coffee beans in grinding roasted coffee beans and contains a fine powder and thin flakes. Not removed, the fine powder and thin flakes may partly remain, but preferably, the fine powder and thin flakes are removed substantially wholly. Regarding the fine powder and thin flakes to be removed in the removing step, those derived from chaff may occupy at least a half of the fine powder and thin flakes, or all the fine powder and thin flakes to be removed therein may be substantially those derived from chaff.

In the production method of the present invention, preferably, the step of removing minor fragments is carried out in a minor fragments removing device to be mentioned in detail hereinunder.

The details of the minor fragments removing device are given in the section of the aroma collecting apparatus of the present invention.

### <Adsorbing Step>

The production method of the present invention includes an adsorbing step of introducing the gas from which minor fragments have been removed into an adsorbent to thereby make the aroma compounds in the gas adsorbed by the adsorbent.

The direction of the gas flowing into and through the absorbent may be at any desired angle relative to the installation surface on which the aroma collecting apparatus is installed (or the ground plane in the case where the aroma collecting apparatus is installed on the ground), and may be, for example, parallel or vertical thereto. In other words, the direction of the gas flowing into and through the adsorbent can be in any desired direction relative to the direction of gravitational force, and can be in any of a substantially opposite direction, nearly the same direction or in an orthogonal direction relative to the direction of gravitational force, or can also be at any other angle. In the present invention, from the viewpoint of increasing the aroma compound trapping performance to thereby increase the adsorption efficiency, preferably, the gas flowing direction is a substantially opposite direction to the direction of gravitational force, and is more preferably, an opposite direction to the direction of gravitational force. In the case where a gas is introduced to run through the adsorbent in a substantially opposite direction to the direction of gravitational force, the volume (bulk volume) of the adsorbent to be used can be smaller than the volume of the bag holder and the aroma compound adsorbing device can be in the state of a so-called fluidized-bed column to thereby reduce the resistance of the adsorbent to the gas flow. In the present invention, from the viewpoint of increasing the aroma compound trapping performance to increase the adsorption efficiency, preferably, the adsorbent is in the state of a fluidized bed in the adsorbing step.

In the production method of the present invention, preferably, a gas flow is generated using a gas flow generating device and the gas from which minor fragments have been removed is introduced into the adsorbent. Using both a flow rate controlling device and a gas flow generating device, the gas flow rate and pressure may be increased. By the combined use, the gas may be made to flow exceeding the resistance of the adsorbent to the gas flow.

The details of the gas flow generating device and the gas flow rate controlling device are described in the section of the aroma collecting apparatus of the present invention given hereinunder.

In the production method, preferably, a guide path having an adsorbent arranged therein may be so arranged as to be branched from the flow channel of the gas from which minor fragments have been removed, so that only a part of the gas from which minor fragments have been removed could be made to flow into and through the guide path and further to flow through the adsorbent to thereby collect aroma compounds.

The details of the guide path are described in the section of the aroma collecting apparatus of the present invention.

Means for introducing the gas from which minor fragments have been removed into the adsorbent so as to make aroma compounds adsorbed by the adsorbent may be any of a batch system or a column system. From the viewpoint of workability, a column system is preferably employed. Regarding adsorbing method using a column system device, for example, the gas is introduced into a column that a bag holding the above-mentioned adsorbent is held so that an aroma compound may be adsorbed by the adsorbent.

By controlling the particle size and the amount of the adsorbent, the bag holder may be made to have some vacant space therein, and further, the gas may be made to flow into or through the adsorbent in a substantially opposite direction to the direction of gravitational force, like in a fluidized-bed column.

The gas amount in introducing the gas from which minor fragments have been removed into the adsorbent is, though not specifically limited thereto, for example, preferably 0.1 to 1000 times by volume of the adsorbent.

Not specifically limited, the temperature of the gas to flow into the adsorbent (this may be referred to as a column inlet port temperature) may be appropriately set depending on the adsorbent amount, the type of the bag and the shape of the bag holder. The temperature of the gas to flow out of the adsorbent (this may be referred to as a column outlet port temperature) is not also specifically limited, but from the viewpoint of increasing the aroma compound adsorption efficiency, the adsorbing step is preferably continued until the column inlet port temperature of the gas and the column outlet port temperature of the gas come to be close to each other (that is, the two temperatures come to be on the same level). For example, the temperature difference between the column inlet port temperature and the column outlet port temperature is preferably 5° C. or less, more preferably 3° C. or less, even more preferably 2° C. or less. In addition, the lapse time for which the column inlet port temperature of the gas and the column outlet port temperature of the gas come to be on the same level is preferably shorter, since the time to be taken until the adsorbent gets dried can be shorter under the condition and the aroma compound adsorption efficiency can be thereby increased.

The flow rate of the gas flowing into the adsorbent (this may also be referred to as a column inlet port flow rate) may be appropriately set depending on the adsorbent amount, the length in the gas flowing direction of the adsorbent part, and the performance of the gas flow generating device and the flow rate controlling device to be mentioned hereinunder, and for example, the flow rate may fall within a range of 1 to 5,000 L/min but is not specifically limited thereto. Concretely, the flow rate of the gas running into the adsorbent (gas running speed) may be 100 to 2,000 L/min, or 150 to 1,000 L/min, or 200 to 800 L/min.

A preferred range of the gas flowing time through the adsorbent may be set depending on the gas flow amount in introducing the gas from which minor fragments have been removed into the adsorbent and on the gas flow rate flowing into the adsorbent.

The linear speed of the gas flowing into the adsorbent (—this may also be referred to as a column inlet port flow rate) may be appropriately set depending on the adsorbent amount, the length of the gas flowing direction in the adsorbent part, the inner diameter of the second flow channel to be mentioned hereinunder, and the performance of the gas flow generating device and the flow rate controlling device also to be mentioned hereinunder, and is not specifically limited. For example, the linear speed is preferably within a range of 0.05 to 10.0 m/s, more preferably within a range of 0.08 to 5.0 m/s, and even more preferably within a range of 0.1 to 4.0 m/s.

Also not specifically limited, the flow rate of linear speed of the gas flowing out of the adsorbent (this may also be referred to as a column outlet port flow rate) is preferably close to the column inlet port flow rate (that is, on the same level). For example, the column inlet port flow rate is 50% or more of the column outlet port flow rate, more preferably 70% or more, and even more preferably 80% or more.

In the adsorption time, the time in which the column inlet port flow rate and the column outlet port flow rate are on the same level is preferably longer, since under the condition, the gas can be introduced evenly into and through the entire adsorbent, the aroma compound adsorption efficiency is high, the gas flow is stable, and the load on the aroma collecting apparatus can be reduced.

### <Step of Controlling Gas Linear Speed>

Preferably, the production method of the present invention includes a step of controlling the linear speed of the gas flowing into the adsorbent, from the viewpoint of realizing adsorbability over the resistance of the adsorbent and from the viewpoint of reducing the load on the gas flow generating device to be mentioned hereinunder, even when a large amount of an adsorbent is held (for example, roughly filled) in the aroma compound adsorbing device.

In the production method of the present invention, the linear speed of the gas to flow into the adsorbent may be controlled using any known gas flow generating device, for example, a suction pump or a blower.

For example, the linear speed of the gas flowing into the adsorbent may be in any desired ratio relative to the linear speed of the gas flowing through the second flow channel, and the ratio may be 100%, 90% or more, 80% or more, 70% or more, 60% or more, 50% or more, 40% or more, 30% or more, 20% or more, 10% or more, 5% or more, or even 1% or more. Concretely, the range includes 0.05 to 35 m/s, 0.08 to 20 m/s, 1.0 to 10 m/s, 1.0 to 5 m/s, or 1.0 to 2 m/s, but is not specifically limited thereto. For example, preferably, the ratio of the linear speed of the gas flowing into the adsorbent relative to the linear speed of the gas flowing into the second flow channel is controlled in accordance with the performance of the gas flow generating device to be mentioned below. According to such controlling, the load on the gas flow generating device can be reduced.

### <Takeout Step>

The production method of the present invention includes a takeout step of taking out the bag from the bag holder. The adsorbent having adsorbed the aroma compounds is taken out of the bag holder of an aroma compound adsorbing device together with the bag that is holding the adsorbent therein. In other words, the bag is taken out from the bag holder together with the adsorbent being held in the bag. By virtue of this, the adsorbent can be prevented from being electrostatically charged and from scattering owing to the charging with static electricity, and consequently, a substantially entire amount of the adsorbent having adsorbed aroma compounds can be taken out of the adsorbent holder. Further, the adsorbent taking out time can be significantly reduced.

The method of taking out the bag from the bag holder is not specifically limited. For example, in the case where one end of the bag (for example, the gas outlet side end) is detachably fixed to the bag holder, the fixed end of the bag is detached from the bag holder and the bag can be taken out of the bag holder. For example, in the case where the strings stringed into the stringing holes Hb1 formed at the gas outlet side end Ha2 of the bag are hung on any desired member (hanging means) in the aroma compound adsorbing device, the mesh lid Ka2 of the bag holder in the aroma compound adsorbing device is opened and the hanging strings can be released to readily take out the bag II holding therein the adsorbent having adsorbed aroma compounds.

### <Collecting Step>

The production method of the present invention includes a collecting step of collecting aroma compounds from the adsorbent to prepare an aroma composition containing the aroma compounds.

In the present invention, in the collecting step, the adsorbent may be held in the bag or may be taken out of the bag in consideration of the handleability and the desired desorption efficiency. In the case where the adsorbent is held in the bag in the collecting step, for example, the bag holding the adsorbent therein is put in a container for desorption operation to be mentioned below (this may also be referred to as a desorption column), and then a desorbent to be mentioned hereinunder may be applied to the desorption column. In the case where the adsorbent is taken out of the bag, for example, the adsorbent is put in an arbitrary container, and a desorbent to be mentioned hereinunder is injected into the container and mixed with the adsorbent to give a slurry, and the slurry may be sucked up with any desired means (pump or the like) and then injected into the above-mentioned desorption column.

Preferably, the collecting step is a step of collecting aroma compounds from the adsorbent to prepare a solution containing the aroma compounds.

In the present invention, preferably, in the collecting step, aroma compounds are desorbed from the adsorbent using an organic solvent. In the collecting step, more preferably, aroma compounds are desorbed from the adsorbent using propylene glycol or ethanol as a desorbent, and the resultant propylene glycol solution or ethanol solution is obtained as an aroma composition, for example, a coffee flavor improver. Also, one prepared by diluting the propylene glycol solution or the ethanol solution, or one prepared by mixing the propylene glycol solution and the ethanol solution may also be used as an aroma composition. In this description, propylene glycol and ethanol may be referred to as a desorbent or a solvent. Before desorbing aroma compounds from the adsorbent using an organic solvent, the adsorbent may be washed with water.

Common organic solvents may be used here, including alcohols, oils and fats.

Examples of alcohols for use in the collecting step include, not specifically limited thereto, alcohols such as methanol, ethanol, n-propanol, isopropanol, butanol, 2-butanol, and t-butanol; ketones such as acetone; and polyalcohols such as ethylene glycol, propylene glycol, glycerin, 1,3-butylene glycol, and 1,2-butylene glycol. One alone or a mixture of two or more of these compounds may be used here either singly or as combined. Among these, alcohols and polyalcohols are preferred. Ethanol and propylene glycol are more preferred. Either singly or as combined, a propylene glycol solution and an ethanol solution may be added to food and drink as an aroma composition. A mixture of the two may be prepared to be an aroma composition and may be added to food and drink. In this description, an aroma composition obtained by desorbing with propylene glycol may be referred to as “PG solution”, and an aroma composition obtained by desorbing with ethanol may be referred to as “ethanol solution”.

The blending ratio of the PG solution and the ethanol solution is any desired one, and for example, the mass ratio of the ethanol solution to the PG solution may be within a range of 0.1 to 10, 0.2 to 5, 0.5 to 3 or 0.8 to 2 relative to 1 part by mass of the PG solution. Also, for example, the mass ratio of the PG solution to the ethanol solution may be about 1/1, about 2/1, about 3/2, about 2/3, or about 1/2. The PG solution enhances the aroma at the top and also enhances the sustainability of the voluminousness and mildness in the middle and the later, while the ethanol solution enhances the voluminousness in the middle and later but especially enhances the aroma at the top, and therefore, the ratio of the PG solution to the ethanol solution can be appropriately controlled in accordance with the desired taste and aroma.

Not adhering to any theory, propylene glycol and ethanol can acetalize a part of the collected aroma compounds (PG acetalization, diethyl acetalization) and, as a result, there is a probability that the flavor improving effect can be thereby increased.

Also in the present invention, the PG solution and the ethanol solution containing aroma compounds can be appropriately diluted with a solvent usable in food and drink, depending on the intended use purpose. Not specifically limited thereto, examples of the solvent include water (e.g., ion-exchanged water), alcohols such as ethanol, polysaccharides such as propylene glycol and glycerin, as well as triacetin, various fatty acids, and vegetable oils and fats. An aqueous alcohol solution of 50 to 100% by mass may be used. For ethanol, hydrous ethanol having an ethanol concentration of 50 to 95% by mass is preferably used; and for PG, 50 to 100 mass % PG is preferably used.

In the case where columns are used, the flow rate of alcohol to flow through a column is preferably SV=0.1 to 20.

The amount of alcohol to be used is not specifically limited, and the flow amount thereof is preferably 1 to 100 times by volume of the adsorbent, more preferably 3 to 40 times, even more preferably 5 to 20 times thereof.

By eluting the aroma compounds adsorbed by the adsorbent with an alcohol or the like, a water-soluble aroma composition (aroma condensate) can be obtained.

Not specifically limited, examples of the fats and oils for use for desorption include vegetable fats and oils such as soybean oil, rice oil, sesame oil, peanut oil, corn oil, rapeseed oil, coconut oil and palm oil, and hardened fats and oils thereof; animal fats and oils such as beef tallow, lard and fish oil, and hardened fats and oils thereof; and middle chain fatty acid triglycerides (hereinafter this may be referred to as MCT). From the viewpoint the stability of the aroma composition to be obtained, MCT is preferred. Examples of MCT include triglycerides of middle acid fatty acid having 6 to 12 carbon atoms, such as caproic acid triglyceride, caprylic acid triglyceride, capric acid triglyceride, lauric acid triglyceride and mixtures thereof. In particular, caprylic acid triglyceride, capric acid triglyceride and mixtures thereof are preferred. These MCT mixtures are inexpensive and are readily available on the market. A mixture of two or more aroma compositions each using different fats and oils may be used as an aroma composition obtained according to the production method of the present invention.

The amount of the fats and oils to be used herein differs depending on the type of the animal or plant material and the aroma compound concentration in the gas. Desorption may be carried out under a static condition, and the desorption temperature and the desorption time may be appropriately selected. For example, a desorption time falling within a range of 5 minutes to 2 hours at a temperature ranging from 10 to 80° C. may be referred to as one example of the operation. After desorption, the resultant desorbed liquid is kept statically, and may be subjected to separation between the oily phase part and the aqueous phase part according to an ordinary separation method of, for example, decantation or centrifugation. A component of fats and oils may be further added to the aqueous phase part for extraction to thereby efficiently collect the intended aroma compounds. The resultant oily phase part may be dewatered, for example, using a dewatering agent such as anhydrous sodium sulfate, and may be filtered according to a clarification filtering means using, for example, filter paper to give an oil-soluble flavor composition.

### <Reuse and Washing of Desorbent>

In the case where the pressures of a liquid introduced into the adsorbent before and after the desorption with an organic solvent are compared and are on the same level (for example, not more than 2 times), it can be judged that clogging has not occurred or on an ignorable level and the adsorbent on that level is reusable without washing. The method of maintaining the aroma collecting apparatus may include a step of comparing the pressures of liquid introduced into the adsorbent before and after the desorption with an organic solvent, and a step of confirming as to whether or not the pressures are on the same level. Specifically, it is preferable that, before and after the desorption, the device is purged with pure water, then ultrapure water is introduced thereinto at SV=10 or so and the pressure of the water flow is measured, and then the ratio of the pressure after the desorption to that before the desorption is calculated.

On the other hand, the production method of the present invention may include an adsorbent washing step. Specifically, the method of maintaining the aroma collecting apparatus may include an adsorbent washing step. In the production method of the present invention, minor fragments are not almost adsorbed by the adsorbent, but any other component contained in the gas (especially a polymerizable component) may be adsorbed by the adsorbent. An adsorbent washing method is well known to those skilled in the art, in which a few kinds of solvents each having a different polarity that varies sequentially are applied to the adsorbent. The kind of the solvent is not specifically limited. For example, an alcohol substance such as PG and/or ethanol is applied to the adsorbent for desorption, and then ethyl acetate and hexane are applied thereto in that order for washing the adsorbent, and in regenerating the adsorbent, ethyl acetate and water may be applied thereto in that order.

Preferably, the adsorbent is reused until the operation of the desorption and collection are repeated for a total of 5 times or more while, if desired, it is washed after collection of aroma compounds therefrom. More preferably, the adsorbent is reused until the operation is repeated for a total of 10 times.

### <Aroma Composition>

The aroma composition produced according to the production method of the present invention contains aroma compounds emitted in fragmenting an animal or plant material, and gives an aroma that is emitted in the fragmenting step.

Specifically, the aroma that is emitted in fragmenting an animal or plant material is preferably an aroma perceivable in fragmenting an animal or plant material, for example, an aroma in grinding them, an aroma in fracturing them, an aroma in mincing them, an aroma in powdering them, or an aroma in flaking them, and these aroma is preferably given in the top. Further, voluminousness is preferably given in the middle and later.

For example, in the case of roasted coffee beans, the aroma compound that is emitted in grinding roasted coffee beans is contained in the aroma composition, and the aroma that is emitted in grinding roasted coffee beans is given by the composition. In addition, when the aroma composition is added to coffee-taste food and drink (for example, coffee drink) as a coffee flavor improver, preferably, it enhances the sweet and roasty aroma given at the top and enhances or improves the flavor in the middle and later (for example, mellows the flavor and enhances the voluminousness), and more preferably, the composition also enhances the flavor sustainability.

Specifically, the aroma that is emitted in grinding roasted coffee beans is preferably the aroma given in grinding roasted coffee beans, and more preferably the aroma at the top is strong, and also the aroma in the middle and later is voluminous and keeps good aftertaste.

In one preferred embodiment of the present invention, in collecting the aroma compounds emitted in grinding roasted coffee beans, propylene glycol and/or ethanol are specifically selected from among various solvents heretofore used in the field of food, thereby exhibiting a significant and diversified flavor enhancing effect, as shown in Examples to be given hereinunder. The aroma composition obtained from roasted coffee beans in the present invention exhibits, as a whole, an excellent flavor improving effect not only for the aroma at the top but also for the aroma in the middle and later, and can improve the flavor of coffee drink in a good balance not heretofore experienced in the art. Not adhering to any theory, the reason may be presumed to be as follows. First, the aroma composition obtained from roasted coffee beans in the present invention contains a highly-volatile aroma compound(s) given at the top in a high proportion, but contains a relatively heavy aroma compound(s) given in the middle and later in an appropriate amount, and consequently, when added to coffee-taste food and drink, the composition can sufficiently increase the light aroma compound(s) (that is, enhance the aroma at the top) that may be lose in producing the food and drink, and can also enhance the flavor in the middle and later, and consequently, it is considered that the aroma composition of the present invention can enhance the entire flavor of coffee-taste food and drink in a good balance not heretofore experienced in the art. Further, in desorbing the aroma compounds from the adsorbent, minor reaction products of a part of the aroma compounds and the desorbent (for example, PG acetal compounds (propylene glycol acetal compounds), diethyl acetal compounds, methyl ester compounds) may be formed and it is considered that these may have some influence on the flavor at the top or in the middle and later.

It is presumed that, owing to the interaction of the above-mentioned points, the present invention can exhibit excellent effects to be mentioned below for coffee-taste food and drink.
The aroma composition enhances the sweet and roasty aroma at the top, and enhances the flavor in the middle and later to give a mild flavor as a whole, and therefore increases flavor sustainability.
The aroma composition gives a sharp aftertaste.
The aroma composition enhances a light and fresh milk-like flavor.

### [Use of Aroma Composition Produced According to Production Method of the Invention]

The aroma composition produced according to the production method of the present invention can be added to various substrates such as foods and drinks, cosmetics, health and hygiene products and medicines.

For example, in the case where roasted coffee beans are used, the aroma composition produced according to the production method of the present invention is preferably used for substrates that give a coffee-like flavor, and more preferably the composition is added to foods and drinks having coffee-like flavor. Further, the aroma composition obtained according to the production method of the present invention can be used as a coffee flavor improver and added to various flavoring compositions that are required to be given a coffee flavor. In the present invention, coffee taste or flavor means an aroma and/or a taste that may evoke coffee or roasted coffee beans.

Preferably, food and drink may contain the aroma composition produced by the production method of the present invention in an amount of 0.01 to 10% by mass relative to the total mass of the food and drink, more preferably 0.05 to 7% by mass. Preferably, the composition may contain the aroma composition produced by the production method of the present invention in an amount of 0.1 to 10% by mass relative to the total mass of the composition to which the aroma composition produced by the production method of the present invention is added, more preferably 0.5 to 5% by mass.

The food and drink are preferably a packed food or drink, more preferably a packed drink. The composition produced according to the production method of the present invention contains a relatively large amount of volatile compounds having a low molecular weight. A packed food or drink containing the aroma composition produced by the production method of the present invention can therefore give a strong top aroma. In addition, the aroma composition can give an aroma in the middle and later (of a relatively poorly volatile compound(s)). Consequently, the packed food or drink that contains the aroma composition produced according to the production method of the present invention can give, when unpacked, a rich aroma that is emitted in fragmenting an animal or plant material, and can also enhance the total flavor of food and drink.

Examples of the packed food and drink include frozen desserts such as ice cream, soft cream and sherbet; confectionery such as biscuit, cookie, rice cracker, steamed yeast bun with filling, chocolate, cream-filled confectionery, jelly, gum, candy and caramel; bread; soup stock from dried bonitos; seasonings such as dressing, spread and jam; flavor oils, frozen foods, and health foods (for example, Foods with Function Claims, dietary supplements, food for specified health use).

Packed drink means a drink having a suitable concentration for drinking and is packed in a container (generally, it is sterilized before and after packed in a container). The packed drink is preferably a packed drink filled in a PET bottle, a can or a paper container. The packed drink includes tea-type drink such as barley tea drink, cereal tea drink, brown rice tea drink, and so-called mixed tea drink prepared by mixing tea and roasted cereal (blend tea drink); tea-type drink such as green tea drink, oolong tea drink, and red tea drink; coffee drink; and beer-taste drink such as beer, low-malt beer, so-called third beer (quasi-beer), nonalcoholic beer-taste drink.

The food and drink may be thermally sterilized. In production of packed drinks, the products are subjected to retort sterilization (thermal sterilization at 121° C. for 10 minutes or so) or UHT sterilization (thermal sterilization at 135° C. for 1 minute or so). However, ordinary aroma at the top is often lost by heating. The aroma composition produced according to the production method of the present invention has a strong aroma at the top and hardly loses the top aroma even when heated, and is therefore favorably used for thermally sterilized food and drink. In addition, consequently, the aroma composition of the present invention is also favorably used even in food and drink that are required to be heated before eating.

[Apparatus for Collecting Aroma from Animal or Plant Material]

The apparatus for collecting aroma from an animal or plant material of the present invention (hereinafter this may be referred to as the aroma collecting apparatus of the present invention) includes:

a device for fragmenting an animal or plant material,

a first flow channel which is communicated with the fragmenting device and through which a gas can flow, together with aroma compounds contained in the gas and minor fragments contained in the gas, which aroma compounds have been emitted in fragmenting the animal or plant material and containing minor fragments,

a minor fragments removing device communicated with the first flow channel,

a second flow channel which is communicated with the minor fragments removing device and through which the gas, from which the minor fragments have been removed, can flow,

an aroma compound adsorbing device communicated with the second flow channel, and

a gas flow generating device that generates a gas flow continuing from the fragmenting device to the aroma compound adsorbing device,

a bag capable of holding an absorbent, and in which:

the aroma compound adsorbing device has a bag holder capable of detachably holding the bag,

the bag holder has a mesh lid at both ends thereof in the gas flowing direction therethrough, and

the bag has pores in a size through which the adsorbent could not pass.

Preferred embodiments of the aroma collecting apparatus of the present invention are described below.

### <Overall Structure of Aroma Collecting Apparatus>

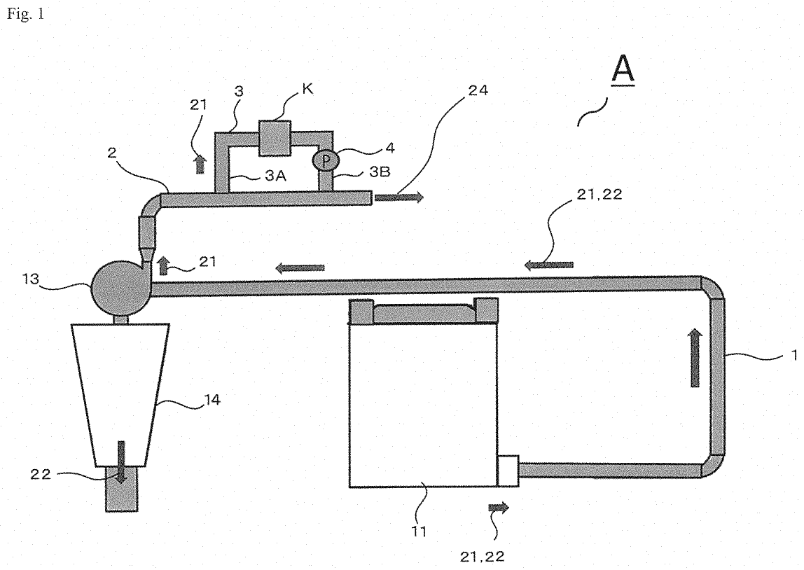

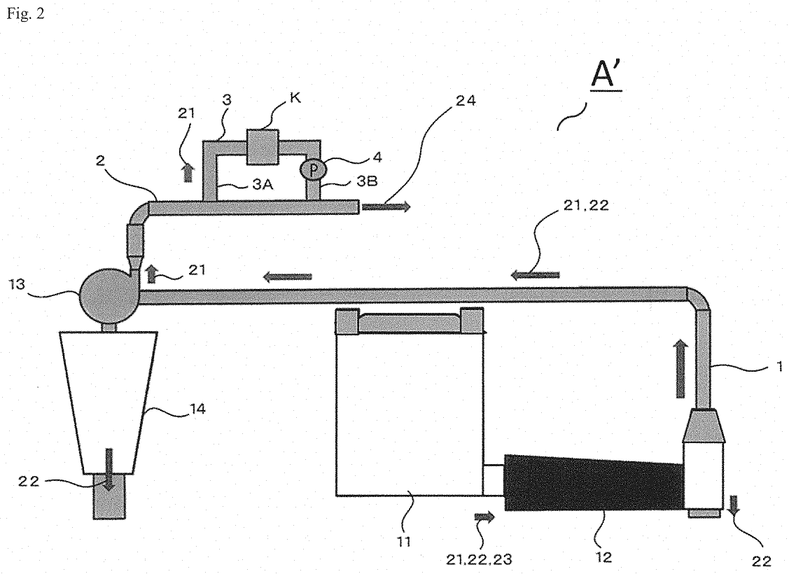

The overall structure of the aroma collecting apparatus is described with reference to the drawings attached hereto. FIG. 1 is a schematic view showing an example of an aroma collecting apparatus of the present invention. FIG. 2 is a schematic view showing another example of an aroma collecting apparatus of the present invention.

One example of the aroma collecting apparatus of the present invention (aroma collecting apparatus A) of FIG. 1 is provided with a fragmenting device 11, a first flow channel 1, a gas flow generating device 13, a minor fragments removing device 14, a second flow channel 2, and an aroma compound adsorbing device K. The aroma compound adsorbing device K has a bag holder Kb having mesh lids Ka1 and Ka2 (for example, see FIG. 3, FIG. 4 or FIG. 5). Further, an example of the aroma collecting apparatus A of FIG. 1 is provided with a guide path 3 and a linear speed controlling device 4, but these are not indispensable components. Also in the present invention, it is desirable that an adsorbent (not shown) is kept held in the bag H arranged in the bag holder Kb.

In the aroma collecting apparatus A of FIG. 1, a fragmenting system having the fragmenting device 11, the first flow channel 1, the gas flow generating device 13, the minor fragments removing device 14 and the second flow channel 2 has been generally used (for example, see the coffee beans grinding device in U.S. Pat. No. 1,649,781 (1927)). In the present invention, the aroma compound adsorbing device K is provided in such an ordinary fragmenting system so as to collect aroma compounds emitted from an animal or plant material in fragmenting the animal or plant material.

In the aroma collecting apparatus A of FIG. 1, while an animal or plant material is fragmented in the fragmenting device 11 to give crude fragmented pieces of an animal or plant material, a gas that contains aroma compounds 21 emitted from the animal or plant material and minor fragments 22 contained in the crude fragmented pieces of the animal or plant material is moved toward the first flow channel 1 from the fragmenting device 11 by the gas flow generated in the gas flow generating device 13. The gas that contains the aroma compounds and the minor fragments 22 moves by the gas flow from the first flow channel 1 to the minor fragments removing device 14. In the minor fragments removing device 14, the gas from which the minor fragments have been removed (but which contains the aroma compounds 21) moves toward the second flow channel 2 while the minor fragments 22 are, after removed in the minor fragments removing device, discharged out of the apparatus. With the gas flow generated in the gas flow generating device 13 (optionally along with the gas flow generated in the linear speed controlling device 4), a part of the gas from which the minor fragments have been removed (but contains the aroma compounds 21) flows from the second flow channel 2 into the guide path 3, and then flows into the adsorbent held in the aroma compound adsorbing device K arranged in the guide path 3, that is, the gas is introduced into the adsorbent so that the aroma compounds 21 is adsorbed by the adsorbent. The gas from which the aroma compounds 21 has been adsorbed by the adsorbent and which has flowed through the adsorbent is again moved to the second flow channel 2 through the outlet port 3B of the guide path, and is combined with the gas which did not flow into the guide path 3 but flow through the second flow channel 2 and from which the minor fragments were removed, and is discharged out of the apparatus as an exhaust gas 24.

In the present invention the guide path 3 is not an indispensable component as so mentioned hereinabove, and therefore, without providing the guide path 3, not a part but all of the gas flowing through the second flow channel (the gas containing the aroma compounds 21 but not containing the minor fragments) may be introduced into the aroma compound adsorbing device K. In this case, the aroma compound adsorbing device K may be arranged in the second flow channel.

Another example of the aroma collecting apparatus of the present invention (aroma collecting apparatus A′) of FIG. 2 is provided with a fragmenting device 11, a minor fragments preremoving device 12, a first flow channel 1, a gas flow generating device 13, a minor fragments removing device 14, a second flow channel 2, and an aroma compound adsorbing device K. The example of the aroma collecting apparatus of FIG. 2 is provided with a guide path 3 and a linear speed controlling device 4, but these are not indispensable components.

In the aroma collecting apparatus of FIG. 2, crude fragmented pieces of an animal or plant material produced by fragmenting the animal or plant material in the fragmenting device 11 are transferred to the minor fragments preremoving device 12 by a transport system not shown. In the minor fragments preremoving device 12, at least a part of the minor fragments 22 are removed from the crude fragmented pieces of the animal or plant material, and the removed minor fragments 22 are housed in a waste chamber not shown and are discharged out of the system. On the other hand, the gas containing the aroma compounds 21 and containing the minor fragments 22 not removed in the minor fragments preremoving device 12 moves toward the first flow channel 1 by the gas flow generated in the gas flow generating device 13. The flow of the aroma compounds 21 and the minor fragments 22 after the first flow channel 1 is the same as in FIG. 1.

Preferred embodiments of the devices which the aroma collecting apparatus of the present invention preferably includes are described hereinunder.

### <Fragmenting Device>

The aroma collecting apparatus of the present invention is provided with a fragmenting device for an animal or plant material.

The fragmenting device is not specifically limited. For example, a roller mill etc. may be used.

The gas to be generated by fragmenting in the fragmenting device 11 is transported to the adsorbent by the gas flow generated in the gas flow generating device and is therefore hardly spread out, and consequently, the fragmenting device is not always required to be closed up. However, from the viewpoint of efficiently collecting the aroma compounds, the fragmenting device 11 may be communicated with the first flow channel 1 and the other parts may be closed up during fragmenting operation.

### <Minor Fragments Preremoving Device>

Preferably, the aroma collecting apparatus of the present invention is further provided with a minor fragments preremoving device between the fragmenting device and the first flow channel.

Preferably, the minor fragments preremoving device is communicated with the fragmenting device to remove at least a part of the minor fragments from the crude fragmented pieces of an animal or plant material obtained by fragmenting an animal or plant material. The purified fragmented pieces of the animal or plant material from which the minor fragments have been removed (that is, a fragmented pieces of the animal or plant material having a desired size) can be used as a food or drink or for production thereof.

A part or most of the minor fragments may be removed and discharged out of the system. When the amount of the minor fragments that move to the first flow channel from the minor fragments preremoving device is smaller, the load on the downstream, i.e., the minor fragments removing device and/or the exhaust system of the aroma collecting apparatus can be reduced more.

Any known device is usable as the minor fragments preremoving device, and a classification device such as a shaking sieve or a wind-driven classifier is preferably used.

### <First Flow Channel>

The aroma collecting apparatus of the present invention is provided with a first flow channel which is communicated with the fragmenting device and through which a gas can flow, together with aroma compounds contained in the gas and minor fragments contained in the gas which aroma compounds have been emitted in fragmenting an animal or plant material.

The first flow channel may be directly communicated with the fragmenting device, or may be communicated with the fragmenting device via the minor fragments preremoving device.

Though not specifically limited, the diameter (inner diameter) of the first flow channel is, from the viewpoint of making more gas flow therethrough, preferably 30 mm or more, more preferably 50 mm or more, even more preferably 100 mm or more, further more preferably 200 mm or more, and especially more preferably 300 mm or more.

The minor fragments preremoving device 12 may be provided with a suction mouth to be coupled with the first flow channel 1.

### <Minor Fragments Removing Device>

The aroma collecting apparatus of the present invention is provided with a minor fragments removing device communicated with the first flow channel to remove minor fragments.

Any known device may be used as the minor fragments removing device, and a cyclone-type separating device (powder separating device) is preferably used.

### <Second Flow Channel>

The aroma collecting apparatus of the present invention is provided with a second flow channel communicated with the minor fragments removing device, through which the gas from which minor fragments have been removed can flow.

In the aroma collecting apparatus of the present invention, the diameter (inner diameter) of the second flow channel is not specifically limited and is preferably 30 mm or more from the viewpoint of making more gas flow therethrough, more preferably 50 mm or more, even more preferably 100 mm or more, further more preferably 200 mm or more, and especially preferably 300 mm or more. The second flow channel can be arranged in any desired manner so that the direction of the gas to run into the aroma compound adsorbing device to be mentioned below can be a desired direction.

### <Aroma Compound Adsorbing Device>

The aroma collecting apparatus of the present invention is provided with an aroma compound adsorbing device communicated with the second flow channel.

The aroma compound adsorbing device has a bag holder capable of detachably holding the bag.

The bag holder has a mesh lid at both ends thereof in the gas flowing direction therethrough.

The bag has pores in a size through which the adsorbent could not pass.

Regarding the arrangement of the aroma compound adsorbing device, the aroma compound adsorbing device is arranged parallel to the installation surface of the aroma collecting apparatus (parallel to the ground contact surface, that is, horizontally) in FIGS. 1 and 2, but the device may also be arranged vertical to the installation surface, or at any other angle thereto. The device may also be so arranged that the direction of the gas flowing into and through the aroma compound adsorbing device can be the direction come close to or go away from the installation surface of the aroma collecting apparatus. In other words, the aroma compound adsorbing device and the direction of the gas flowing into and through the adsorbent can be substantially opposite to or substantially the same as the direction of gravitational force, or can also be perpendicular thereto, or may be at any other angle. Preferably, the gas flows at an angle within 30°, within 200, within 10° or within 50 relative to the direction of gravitational force, or in the direction opposite to the direction of gravitational force. In the present invention, from the viewpoint of enhancing the aroma compound trapping performance to thereby enhance the adsorption efficiency, the aroma compound adsorbing device is arranged preferably in such a manner that the direction of the gas flowing into and through the aroma compound adsorbing device and the adsorbent can be in a substantially opposite direction to the direction of gravitational force. The diameter of the aroma compound adsorbing device is not specifically limited. From the viewpoint of enhancing the gas flowing or introduction efficiency and reducing the load on the fragmenting device, the diameter is preferably larger, and is, for example, preferably 10 mm or more, more preferably 20 mm or more.

### <Gas Flow Generating Device>

The aroma collecting apparatus of the present invention is provided with a gas flow generating device capable of generating a gas flow continuing from the fragmenting device to the aroma compound adsorbing device. The gas flow generating device 13 can generate a gas flow that continues through the fragmenting device 11, (the minor fragments preremoving device 12), the first flow channel 1, the minor fragments removing device 14, the second flow channel 2 and the aroma compound adsorbing device K.

The gas flow generating device may be a blower or a suction aspirator.

Examples of a suction aspirator include a suction blower.

### <Guide Path>

Preferably, the aroma collecting apparatus is provided with a guide path 3 branched from the flow channel for the gas from which the minor fragments have been removed, i.e., the second flow channel, and communicated with the aroma compound adsorbing device. This configuration is made in order to introduce only a part of the gas from which the minor fragments have been removed into the guide path and the adsorbent to collect aroma compounds for suppressing the adsorbent resistance. In that manner, the aroma compound adsorbing device may be communicated with the second flow channel via the guide path.

The diameter (inner diameter) of the guide path is, though not specifically limited, preferably 5 mm or more as the inner diameter from the viewpoint of more gas can flow through the guide path, more preferably 15 mm or more, even more preferably 30 mm or more, further more preferably 50 mm or more, even further more preferably 70 mm or more, still further more preferably 100 m or more, still further more preferably 150 mm or more, still further more preferably 200 mm or more, and especially more preferably 300 mm or more.

The guide path 3 may be formed integrally with the second flow channel, or may be detachably connected to the second flow channel. At least a part of the guide path 3 may be fixed to the second flow channel 2 by means of any desired fixing means such as an adhesive tape or screws.

The inlet port 3A of the guide path 3 may be branched at any position of the second flow channel 2. For example, in FIG. 1, the port is arranged at the position extending horizontally (in the right and left direction on the paper) from the second flow channel 2, but may also be arranged in the second flow channel 2 extending in the vertical direction (in the upper direction on the paper) from the gas flow generating device 13.

Preferably, the outlet port 3B of the guide path 3 is connected to the second flow channel 2 so that the gas after the adsorption of the aroma compounds can be returned back to the second flow channel 2.

The inlet port 3A and the outlet port 3B of the guide path 3 each may be connected to the second flow channel 2 at any angle, and the guide path 3 may be linear, or curved, or may be folded at one or more position.

The material of the guide path 3 is not specifically limited, and may be made of, for example, a metal or a resin.

FIGS. 1 and 2 disclose embodiments each having one guide path 3 and one aroma compound adsorbing device K. However, when a larger amount of aroma compounds is desired to be adsorbed, plural guide paths 3 and plural aroma compound adsorbing devices K may be arranged in the apparatus.

### <Linear Speed Controlling Device>

Preferably, the aroma collecting apparatus of the present invention is further provided with a linear speed controlling device 4 for controlling the linear speed of the gas from which the minor fragments have been removed.

The linear speed controlling device may be a blower or a suction aspirator. Examples thereof include a blower fan and a suction pump.

The position of the linear speed controlling device in the aroma collecting apparatus of the present invention is not specifically limited, and depending on the type thereof, the device may be arranged either upstream or downstream of the gas flow with respect to the aroma compound adsorbing device. For example, a blower may be arranged at the upstream, and a suction aspirator may be arranged at the downstream.

The suction aspirator to be used as the linear speed controlling device 4 is preferably one having a higher pumping performance than that of the gas flow generating device 13 from the viewpoint of efficiently collecting an aroma compound. Preferably, the linear speed controlling device 4 is arranged in the guide path 3. The linear speed controlling device 4 may be arranged at the inlet port 3A of the guide path, or at the outlet port 3B of the guide path.

### EXAMPLES

The present invention is described more specifically with reference to the following Examples and Comparative Examples. In the following Examples, the material used, its amount and ratio, the details of the treatment and the treatment process may be suitably modified or changed not overstepping the spirit and the scope of the invention. Accordingly, the invention should not be limitatively interpreted by the Examples mentioned below.

### Examples 1 and 2, Comparative Example 1

### (1) Adsorption Test

An outline of the aroma collecting apparatus used in Examples 1 and 2, and Comparative Example 1 is shown in FIG. 2. Specifically, the aroma collecting apparatus is proved with a fragmenting device 11, a minor fragments preremoving device 12, a first flow channel 1, a gas flow generating device 13, a minor fragments removing device 14, a second flow channel 2, a guide path 3, and an aroma compound adsorbing device K. In FIG. 2, the aroma compound adsorbing device K is described to be parallel to the installation surface of the aroma collecting apparatus (parallel to the ground contact surface, that is, horizontally), but herein, the aroma compound adsorbing device K was arranged substantially vertically to the installation surface, and the gas flowing direction in the adsorbent part was made to be in an opposite direction to the direction of gravitational force (that is, in a vertical upward direction).

The aroma collecting apparatus A is provided with a roller mill as the fragmenting device 11. The fragmenting device 11 is communicated with the minor fragments preremoving device 12, and the other parts can be kept closed during fragmenting. The minor fragments preremoving device 12 is communicated with the fragmenting device 11.

As the minor fragments preremoving device 12, used here was a vibrating classifier equipped with a sieve (opening 0.8 mm), and the first flow channel 1 is communicated with the minor fragments preremoving device 12 and the gas flow generating device 13. The gas flow generating device 13 is communicated with the first flow channel 1 and the second flow channel 2.

The aroma collecting apparatus is provided with a suction blower as the gas flow generating device 13. The suction blower can generate a gas flow that continues through the fragmenting device 11, the minor fragments preremoving device 12, the first flow channel 1, the minor fragments removing device 14, the second flow channel 2 and the aroma compound adsorbing device K. Note that the apparatus may be provided with a suction pump as the linear speed controlling device 4 downstream the gas flow after the aroma compound adsorbing device K to be able to generate a gas flow along with the gas flow generating device 13, but a suction pump is not used in this example.

The aroma collecting apparatus is provided with a cyclone-type separating device as the minor fragments removing device 14.

The first flow channel 1 and the second flow channel 2 each have an inner diameter of 200 mm.

The aroma collecting apparatus is provided with the aroma compound adsorbing device K in the guide path 3 branched from the second flow channel 2 having an inner diameter of 200 mm. The apparatus was so planned that the opening of the damper to lead to the second flow channel after the inlet port 3A of the guide path could be 5% and the guide path 3 could accept gas flowing thereinto. The entire amount of the gas having flowed into the guide path 3 flows into the aroma compound adsorbing device K.

The aroma compound adsorbing device K used in Example 1 has a configuration shown in FIG. 4. Specifically, the configuration of the device is as follows.
Mesh lids Ka1 and Ka2: Made of 150-mesh SUS (stainless steel).
Bag holder Kb: Made of an acrylic resin (inner diameter 190 mm, outer diameter 200 mm, length 540 mm). A cylindrical sidewall type with no pores in the sidewall.
Gas direction adjuster Kc: As the gas direction adjuster Kc, a doughnut-shaped baffle plate having a circular hole having a diameter of 170 mm (see FIG. 5, FIG. 6(C)) was arranged between the bag holder Kb and the mesh lid Ka1 to accept gas introduction into the inside of the bag H through the hole. A SUS plate for pressing the mesh lid Ka1 was additionally arranged.
Adsorbent amount: 2 kg.
Adsorbent (not shown): Sepabeads SP70 (aromatic synthetic adsorbent, manufactured by Mitsubishi Chemical Corporation). For preventing cracking thereof, the adsorbent was made to absorb pure water, and then put into the aroma compound adsorbing device, before completely dried. The initial water content was 55 to 65% by mass.
Bag H: Cylindrical mesh bag made of 200-mesh nylon. Length 720 mm, cross-sectional diameter 185 mm.
Gas inlet side end Ha1 of bag: Closed as shown in FIG. 7(A).
Gas inlet side end Ha2 of bag: At the gas outlet side end Ha2 of the bag, a string passage Hb2 having stringing holes Hb1 having a width of 20 mm is formed. After an adsorbent has been put into the bag from the gas outlet side end Ha2, strings Hb3 are introduced to pass through the string passage Hb2, then drawn and tightened to drawn and gather the side of the bag to thereby close the gas outlet side end Ha2 to envelop the adsorbent in the bag. With that, strings Hb3 are hooked on a hooking means (not shown) of the bag holder Kb to fix the bag H inside the bag holder Kb (see FIG. 7(A) and FIG. 4). The volume of the adsorbent is about 20% of the volume of the bag.
Adsorption time: About 10 hours a day.

The aroma compound adsorbing device K used in Example 2 has a configuration shown in FIG. 5. Specifically, the following points were added to the aroma compound adsorbing device used in Example 1.
Gas inlet side end Ha1 of bag: The bag is provided with a weight holding part Hc1 having a width of 60 mm to extend from the gas inlet side end Ha1 of the bag, and a string passage Hb2 having a width of 20 mm in which strings Hb3 introduced thereinto through the stringing holes Hb1 pass, as shown in FIG. 7(B) to (D). Further, a weight Hc2 is put in the weight holding part Hc1, and then the strings Hb3 introduced through the stringing holes Hb1 into the string passage Hb2 are drawn to gather the side of the bag to thereby envelop the weight Hc2 in the resultant bag H (see FIGS. 7(C) and (D)).
Weight Hc2: Ring made of SUS (outer diameter 185 mm, inner diameter (hole diameter) 170 mm, about 500 g).

The aroma compound adsorbing device K used in Comparative Example 1 is the same as that used in Example 1 in Japanese Patent 6184627, which differs from the aroma compound adsorbing device shown in FIG. 3 in that it does not have the bag H and an adsorbent is directly put in the bag holder Kb.

Each of the aroma collecting apparatus as above was used, and roasted coffee beans were selected as the animal or plant material, and aroma compounds emitted in fragmenting (grinding) the roasted coffee beans were collected. Specifically, aroma compounds were collected according to the following method.

While a gas flow was kept generated in the gas flow generating device 13, roasted coffee beans (L value: 24) were ground at a rate of 100 kg/h into a ground size of about 1 mm, using a roller mill (fragmenting device 11), thereby giving crude fragmented pieces of the animal or plant material 23 containing minor fragments (in the examples, a crude ground powder of roasted coffee beans).

Using each aroma collecting apparatus, a part of the minor fragments 22 (in the examples and comparative example, those containing a finely ground powder derived from roasted coffee beans and a fine powder and thin flakes derived from chaff) were removed from the crude ground powder of roasted coffee beans (crude fragmented pieces of the animal or plant material 23) and discarded in the classifier (minor fragments preremoving device 12). The minor fragments 22 (mainly containing a fine powder and thin flakes derived from chaff) not removed in the minor fragments preremoving device 12 were, along with the gas in the fragmenting device 11 where the roasted coffee beans were being ground (the gas containing aroma compounds 21), made to flow through the first flow channel 1 continuing from the minor fragments preremoving device 12 by the action of the gas flow. By removing the minor fragments 22 from the crude ground powder of roasted coffee beans (crude fragmented pieces of the animal or plant material 23), a purified powder of roasted coffee beans ground into a desired size can be obtained, and this can be stored in a storing chamber not shown, then taken out of the aroma collecting apparatus, and kept stored in the chamber until use for production for coffee products.

The minor fragments 22 were removed from the gas that contains aroma compounds 21 emitted from roasted coffee beans in grinding roasted coffee beans and contains minor fragments 21, in a cyclone-type powder separator (minor fragments removing device 14).

The minor fragments 22 removed from the gas were stored in a waste chamber (not shown) communicated with the minor fragments removing device 14, and then discarded.

On the other hand, the gas from which minor fragments 22 had been removed was made to flow through the second flow channel 2 communicated with the minor fragments removing device 14. At this time, the opening of the damper attached to the downstream of the minor fragments removing device 14 was appropriately controlled to fall in a range of 0.2 to 0.5 kPa or so.

During grinding the roasted coffee beans, the gas having flowed into the guide path 3 was then introduced to flow through the adsorbent held in the adsorbent holder in the aroma compound adsorbing device, whereby the aroma compounds 21 contained in the gas were adsorbed by the adsorbent.

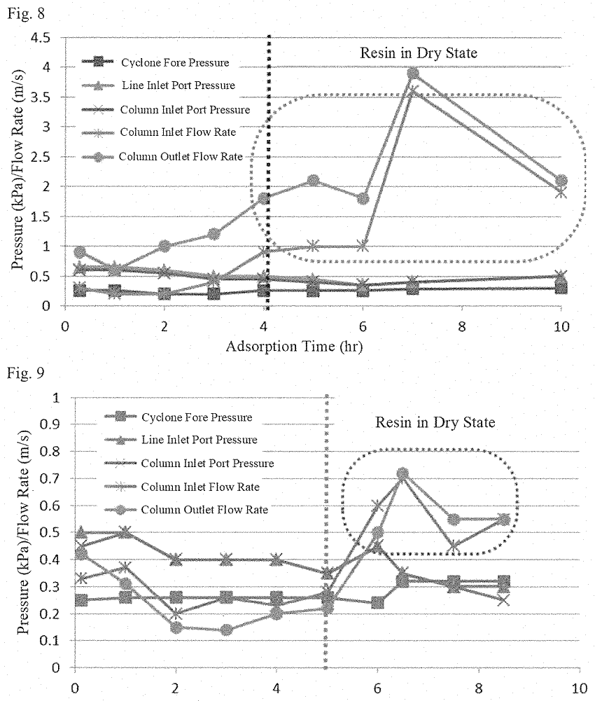

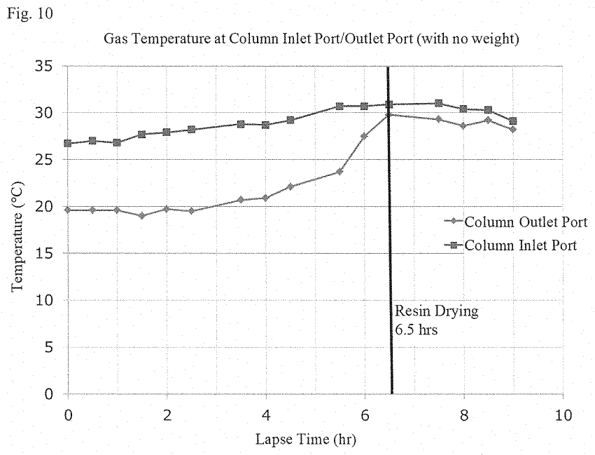

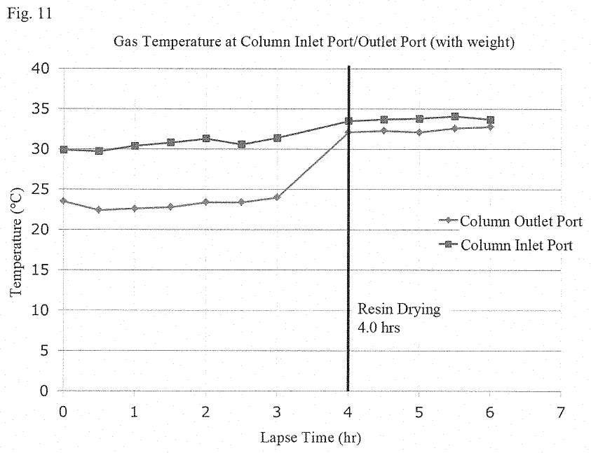

The pressure (cyclone fore pressure) in the upstream of the minor fragments removing device 14, the pressure at the inlet port of the guide path 3 (line inlet port pressure), the pressure and the flow rate at the gas inlet port of each aroma compound adsorbing device K (hereinafter these may be referred to as column inlet port pressure and column inlet port flow rate), and the flow rate at the gas outlet port of each aroma compound adsorbing device K (hereinafter this may be referred to as column outlet port flow rate) were measured (FIGS. 8 and 9). In addition, the gas temperatures at the gas inlet port of the column (column inlet port) and the gas outlet port thereof (column outlet port) were measured (FIGS. 10 and 11).

Observations of the bag holder in the adsorbing step in Examples 1 and 2, and measurements shown in FIGS. 8 to 11 are described below.

### (1-1) Observations

The water content at the start of gas introduction into the adsorbent was 55 to 65% by mass, and the flowability of the adsorbent from the start of adsorption (gas introduction) to the initial stage was low. In a state where the flowability of the adsorbent is low, channeling, which means formation of flow channels only in a part of the adsorbent held in the bag, tends to occur and the adsorbent could not be dried sufficiently and is kept poorly flowable so that the adsorbent in the condition could not sufficiently adsorb aroma compounds.

However, in Examples 1 and 2, the adsorbent was fully dried by the aroma compound-containing gas that flowed through the adsorbent so that the water content in the adsorbent was reduced to 3% by mass and the flowability of the resultant adsorbent was thereby increased.

Here, when the gas flow rate through a dry adsorbent increases, the adsorbent may congregate at the top of the bag (at the gas outlet side end Ha2) so that there may be a possibility that the pressure at each pressure gauge may increase owing to clogging. However, in this case, since the gas escaped through the meshes of the bag, the top of the bag was not completely clogged, and long-term continuous adsorption was possible.

However, in Example 1 using the aroma compound adsorbing device shown in FIG. 4, the gas flow rate increased when the adsorbent was dry and the adsorbent began to congregate at the top of the bag. As a result, the bottom of the bag (gas inlet side end Ha1) lifted up, and a gas flow formed along the outer side surface of the lifted bag, then a part of the gas containing aroma compounds 21 began to flow into the space between the bag and the bag holder with the result that only a part of the gas containing aroma compounds 21 was in contact with the adsorbent in the bag. In addition, the gas flow rate at the column outlet port increased (see FIG. 8 to be mentioned hereinunder), and a turbulence flow would have occurred.

As opposed to this, in Example 2 using the aroma compound adsorbing device shown in FIG. 5, the bag H did not lift up, and any gas flow to flow into the space between the bag H and the bag holder Kb was not observed. This may be because, since the weight Hc2 was arranged in the bag, the bag H did not deform even when given a gas flow, and in addition, the gas flow was almost entirely introduced into the bag owing to the gas direction adjuster Kc (baffle plate). This configuration maintained a state where almost all the gas containing aroma compounds 21 continuously was in contact with the adsorbent.

The above confirms that even the device having the configuration of FIG. 4 can sufficiently adsorb aroma compounds, and can exhibit the effect of the present invention (capable of collecting the aroma emitted in fragmenting an animal or plant material and excellent in the handleability of the adsorbent having adsorbed an aroma compound) but the configuration of FIG. 5 is preferred.

(1-2) Measurements Shown in FIGS. 8 and 9 FIG. 8 is a graph showing a relationship between the adsorbing time (lapse time from the start of gas introduction) and the pressure at different sites in an aroma collecting apparatus or the gas flow amount (at the column inlet port and the column outlet port) therein, in the adsorbing step in Example 1 (not using the weight Hc2); and FIG. 9 is a graph showing that relationship in Example 2 (using the weight Hc2).

As compared with the data of Example 1 shown in FIG. 8, the time for which the column inlet port flow rate and the column outlet port flow rate are on the same level was longer in Example 2 shown in FIG. 9 (using the weight Hc2). This may be because, since a nearly the entire amount of the gas having flowed into the column could flow into the bag H and since the gas could flow almost uniformly in the whole adsorbent in the bag, the gas flow inside the column could be stabilized. Specifically, it can be said that, as compared with that in the case of Example 1 (not using the weight Hc2), the gas could evenly and entirely flow through the whole of the adsorbent in Example 2 using the weight Hc2, and therefore the aroma compound adsorption efficiency was high and the load on the aroma collecting apparatus could be reduced as the gas flow therein was stabilized in Example 2.

### (1-3) Measurements Shown in FIGS. 10 and 11

FIG. 10 is a graph showing a relationship between the lapse time (adsorbing time) from the start of gas introduction and the column inlet/outlet port temperature in the adsorbing step in Example 1 (not using the weight Hc2); and FIG. 11 is a graph showing that relationship in Example 2 (using the weight Hc2).

In the present invention, the vaporization heat to be generated in drying the adsorbent lowers the adsorbent temperature and the temperature of the exhaust gas 24 in the downstream of the aroma compound adsorbing device, and consequently, by measuring the temperature of the exhaust gas 24 with an IR thermometer or the like, it is possible to confirm as to whether or not the adsorbent is in a wet state or in a dry state in continuous operation of the adsorbing step. Here, when the column inlet port temperature of the gas is closer to the column outlet port temperature of the gas, it means that the vaporization heat to be generated in vaporization of water contained in the adsorbent through gas introduction thereinto reduces, that is, the adsorbent is dried by gas introduction thereinto. Accordingly, it is considered that, when the drying time for the adsorbent is shorter, that is, when the adsorbent dries rapidly to release the water having covered the adsorbent, then the time and amount of the gas contact with the adsorbent may increase and the aroma compound adsorption efficiency can be thereby high.

As shown in FIGS. 10 and 11, the gas temperature at the column outlet port and the gas temperature at the gas column outlet port are close to each other in Example 2 using the weight Hc2, as compared with those in Example 1 (not using the weight Hc2), and in addition, the time taken until the adsorbent get dried is shorter in Example 2 than that in Example 1, and accordingly, it is considered that the aroma compound adsorption efficiency in Example 2 is high.

### (2) Takeout Test

In Comparative Example 1, the mesh lid Ka2 of the bag holder in the aroma compound adsorbing device was opened and the adsorbent having adsorbed aroma compounds was taken out. In Comparative Example 1, the adsorbent having adsorbed aroma compounds was electrostatically charged and scattered over the inner wall of the bag holder owing to the charging with static electricity in the takeout step, and therefore it was difficult to take out the adsorbent and much time was taken for taking out the adsorbent. Inevitably, some adsorbent remained in the device.

On the other hand, in Examples 1 and 2, the mesh lid Ka2 of the bag holder in the aroma compound adsorbing device was opened, then the string hooks were removed from the hooking means of the bag holder, and the bag holding the adsorbent having adsorbed aromatic compounds was taken out. In the case of taking out the adsorbent together with the bag containing the adsorbent therein (in other words, taking out the bag together with the adsorbent being held in the bag), the adsorbent did not scatter in the takeout step, and the adsorbent can be rapidly taken out with ease, and consequently, a substantially entire amount of the adsorbent was taken out.

(3) Collection Test from Adsorbent

In Examples 1 and 2, two jacketed 3-liter glass adsorption columns were connected and water at 30° C. was circulated through the jackets. Under the desorption columns, a tube pump (MASTERFLEX US, MODEL 7518-12) for sucking organic solvent was arranged to construct a collecting apparatus from the adsorbent.

The adsorbent having adsorbed aroma compounds was, while kept held in the bag, transferred into the desorption column for desorbing the aroma compounds from the adsorbent.

Subsequently, 12 kg of propylene glycol (PG) kept heated at 30° C. was introduced into the adsorbent from the top of the desorption columns in a down flow mode at SV=2.5 (in the vertical downward direction) and the adsorbent was thus immersed with PG until PG could permeate thereinto. Specifically, PG was poured toward the adsorbent held in the bag, and PG then flowed down through the twisted part of the bag, and in the initial stage, PG could not permeate the adsorbent. Then PG began to gradually permeate the adsorbent, which was confirmed visually. Then, after a necessary amount of PG to permeate the adsorbent was added, the cock at the bottom of the jacket was closed and the adsorbent was kept immersed with PG for 15 minutes.

After immersion of the adsorbent, the cock at the bottom of the desorption column was opened, and PG was begun to be sucked through a tube pump so that the aroma compounds 21 were desorbed from the adsorbent. With PG being added from the top of the jacket as needed, the desorption was continued at 4.2 kg/hr (0.07 kg/min) relative to the targeted SV=2.5 (5 kg/hr). SV (space velocity) is a unit indicating how many times by volume of a desorbent relative to resin (adsorbent) is made to flow per one hour. In 2 hours and 30 minutes after the start of desorption, the desorption was finished, and 10 kg of an aroma composition, 5 times by volume the adsorbent, was collected.

In that manner, a roasted coffee bean flavorings (aroma compositions as a PG solution) containing the aroma compounds trapped in the aroma compound adsorbing device K were obtained.

In Comparative Example 1, 25 kg of propylene glycol (PG) was introduced into the adsorbent at SV=10 according to the method described in [0073] in Japanese Patent 6184627 to desorb the aroma compounds 21 from the adsorbent.

Desorption of the aroma compounds from the adsorbent in the collecting step from the adsorbent was confirmed according to the following method.

In Examples 1 and 2, the inside of the bag was visually observed after the collecting step, and any uneven coloration of the adsorbent (resin) was not seen. As evaluation, the adsorbents in Examples 1 and 2 were visually compared with the adsorbent after the collecting step in Comparative Example 1. As a result, there was not found any significant difference in the condition of the absorbents after the collecting step between the absorbents in Comparative Example 1, and the Examples 1 and 2, and it was judged that the aroma compounds were fully desorbed from the adsorbents.

Accordingly, it is confirmed that, in the case where the adsorbent is processed in the collecting step together with the bag that is holding the adsorbent therein as in Examples 1 and 2, the adsorbent does not scatter in the collecting step, and the aroma compounds can be fully collected in these cases.

From the results of the adsorption test (1), the takeout test (2) and the collection test (3) from the adsorbent, it is confirmed that, according to the production method of the present invention, the handleability of the adsorbent having adsorbed aroma compounds is excellent.

Further, according to the preferred embodiments of the production method of the present invention described in detail hereinabove, it is confirmed the present invention can satisfy both excellent handleability of an adsorbent having adsorbed aroma compounds and excellent collection performance for the aroma compounds.

### REFERENCE SIGNS LIST

1 First Flow Channel
2 Second Flow Channel
3 Guide Path
3A Inlet Port of Guide Path
3B Outlet Port of Guide Path
4 Linear Speed Controlling Device
11 Fragmenting Device
12 Minor Fragments Preremoving Device
13 Gas Flow Generating Device
14 Minor Fragments Removing Device
21 Aroma Compounds
22 Minor Fragments
23 Crude Fragmented Pieces of Animal or Plant Material
24 Exhaust Gas
A One Embodiment of Aroma Collecting Apparatus
A′ Another Embodiment of Aroma Collecting Apparatus
K Aroma Compound Adsorbing Device
Ka1, Ka2 Mesh Lid
Kb Bag Holder
Kc Gas Direction Adjuster
H Bag
Ha1 Gas Inlet Side End
Ha2 Gas Outlet Side End
Ha3 Weight Taking-in and Taking-out Mouth
Hb1 Stringing Holes
Hb2 String Passage
Hb3 String
Hc1 Weight Holding Part
Hc2 Weight

The present disclosure relates to the subject matter contained in Japanese Patent Application No. 2018-124801, filed on Jun. 29, 2018, the contents of which are expressly incorporated herein by reference in their entirety. All the publications referred to in the present specification are also expressly incorporated herein by reference in their entirety.

The foregoing description of preferred embodiments of the invention has been presented for purposes of illustration and description, and is not intended to be exhaustive or to limit the invention to the precise form disclosed. The description was selected to best explain the principles of the invention and their practical application to enable others skilled in the art to best utilize the invention in various embodiments and various modifications as are suited to the particular use contemplated. It is intended that the scope of the invention not be limited by the specification, but be defined claims set forth below.

What is claimed is:

1. A method for producing an aroma composition from an animal or plant material, comprising:
a holding step of holding an adsorbent in a bag and putting the bag in a bag holder inside an aroma compound adsorbing device,
a step of fragmenting an animal or plant material to give crude fragmented pieces of the animal or plant material that contain minor fragments,
a step of removing the minor fragments from a gas that contains aroma compounds emitted from the animal or plant material in fragmenting the animal or plant material and contains the minor fragments,
an adsorption step of introducing the gas from which the minor fragments have been removed into the adsorbent to thereby make the aroma compounds adsorbed by the adsorbent,
a takeout step of taking out the bag from the bag holder, and
a collecting step of collecting the aroma compounds from the adsorbent to prepare an aroma composition containing the aroma compounds, wherein:
the bag holder has a mesh lid at both ends thereof in the gas flowing direction therethrough, and
the bag has pores in a size through which the adsorbent could not pass.

2. The method for producing an aroma composition from an animal or plant material according to claim 1, wherein the adsorbent is one or more selected from a styrene-divinylbenzene copolymer, an ethylvinylbenzene-divinylbenzene copolymer, a 2,6-diphenyl-9-phenyl oxide polymer, a condensation polymer of a methacrylic acid and a diol, and a modified silica gel.

3. The method for producing an aroma composition from an animal or plant material according to claim 1, wherein the gas flowing direction is substantially an opposite direction to the direction of gravitational force.

4. The method for producing an aroma composition from an animal or plant material according to claim 3, wherein the bag is provided with a weight and the weight is arranged around the peripheral part at the end of the gas inlet side of the bag.

5. The method for producing an aroma composition from an animal or plant material according to claim 1, wherein the end part of the gas outlet side of the bag is drawn and gathered to form a drawstring bag or pouch-like shape.

6. The method for producing an aroma composition from an animal or plant material according to claim 1, wherein:
the bag holder has a gas direction adjuster on the gas inlet side, and
the gas is introduced into the inside of the bag via the gas direction adjuster.

7. The method for producing an aroma composition from an animal or plant material according to claim 1, wherein the adsorbent is in the state of a fluidized bed in the adsorption step.

8. The method for producing an aroma composition from an animal or plant material according to claim 1, wherein the aroma compounds are desorbed from the adsorbent using an organic solvent in the collection step.

9. The method for producing an aroma composition from an animal or plant material according to claim 1, wherein the adsorbent is kept held in the bag in the collecting step.

10. An apparatus for collecting aroma from an animal or plant material, which is provided with:
a device for fragmenting an animal or plant material,
a first flow channel which is communicated with the fragmenting device and through which a gas can flow, together with aroma compounds contained in the gas and minor fragments contained in the gas, which aroma compounds have been emitted in fragmenting the animal or plant material,
a minor fragments removing device communicated with the first flow channel,
a second flow channel which is communicated with the minor fragments removing device and through which the gas, from which the minor fragments have been removed, can flow,
an aroma compound adsorbing device communicated with the second flow channel, and
a gas flow generating device that generates a gas flow continuing from the fragmenting device to the aroma compound adsorbing device,
a bag capable of holding an absorbent, and in which:
the aroma compound adsorbing device has a bag holder capable of detachably holding the bag,
the bag holder has a mesh lid at both ends thereof in the gas flowing direction therethrough, and
the bag has pores in a size through which the adsorbent could not pass.

11. The apparatus for collecting aroma from an animal or plant material according to claim 10, wherein the gas flowing direction is substantially an opposite direction to the direction of gravitational force.

12. The apparatus for collecting aroma from an animal or plant material according to claim 11, wherein the bag is provided with a weight and the weight is arranged around the peripheral part at the end of the gas inlet side of the bag.

13. The apparatus for collecting aroma from an animal or plant material according to claim 10, wherein the end part of the gas outlet side of the bag is drawn and gathered to form a drawstring bag or pouch-like shape.

14. The apparatus for collecting aroma from an animal or plant material according to claim 10, wherein:
the bag holder has a gas direction adjuster on the gas inlet side, and
the gas is introduced into the inside of the bag via the gas direction adjuster.

15. The apparatus for collecting aroma from an animal or plant material according to claim 10, wherein the adsorbent is held in a bag.
